(19) United States
(12) Patent Application Publication
(10) Pub. No.: US 2014/0100190 A1
(43) Pub. Date: Apr. 10, 2014

(54) USE OF LIPID CONJUGATES IN CYSTIC FIBROSIS AND APPLICATIONS THEREOF

(71) Applicants: Columbia University, New York, NY (US); Yissum Research Development Company of The Hebrew University of Jerusalem LTD, Jerusalem (IL)

(72) Inventors: Saul Yedgar, Jerusalem (IL); Alice Prince, Larchmont, NY (US); Yuval Cohen, Brooklyn, NY (US)

(73) Assignees: Columbia University, New York, NY (US); Yissum Research Development Company of The Hebrew University of Jerusalem LTD, Jerusalem (IL)

(21) Appl. No.: 14/071,578

(22) Filed: Nov. 4, 2013

Related U.S. Application Data
(63) Continuation of application No. 11/496,728, filed on Aug. 1, 2006.
Application No. 11/496,728 is a continuation-in-part of application No. 10/919,523, filed on Aug. 17, 2004.
Application No. 10/919,523 is a continuation-in-part of application No. 10/790,182, filed on Mar. 2, 2004, now Pat. No. 7,141,552.
Application No. 10/790,182 is a continuation-in-part of application No. 09/756,765, filed on Jan. 10, 2001, now Pat. No. 7,034,006.
(60) Provisional application No. 60/704,874, filed on Aug. 3, 2005.
(60) Provisional application No. 60/780,379, filed on Mar. 9, 2006.

(51) Int. Cl. A61K 47/48

(52) U.S. Cl. CPC A61K 47/4823
USPC 514/59; 514/54

(57) ABSTRACT

This invention provides for the use of compounds represented by the structure of the general formula (A):

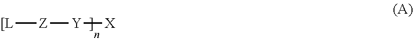

wherein L is a lipid or a phospholipid, Z is either nothing, ethanolamine, serine, inositol, choline, or glycerol, Y is either nothing or a spacer group ranging in length from 2 to 30 atoms, X is a physiologically acceptable monomer, dimer, oligomer, or polymer, wherein X is a glycosaminoglycan; and n is a number from 1 to 1000, wherein any bond between L, Z, Y and X is either an amide or an esteric bond in treating a subject suffering from cystic fibrosis, reducing or delaying the mortality of a subject suffering from cystic fibrosis or ameliorating symptoms associated with cystic fibrosis.

### CROSS-REFERENCE TO RELATED APPLICATIONS

This application is a continuation of U.S. application Ser. No. 11/496,728, filed Aug. 1, 2006. This application is a continuation-in-part of U.S. application Ser. No. 10/919,523, filed Aug. 17, 2004, which is a continuation-in-part of U.S. application Ser. No. 10/790,182, filed Mar. 2, 2004, now U.S. Pat. No. 7,141,552, which is a continuation-in-part of U.S. application Ser. No. 09/756,765, filed Jan. 10, 2001, now U.S. Pat. No. 7,034,006. This application also claims the benefit of United States Provisional is Application Ser. No. 60/704,874, filed Aug. 3, 2005 and U.S. Provisional Application Ser. No. 60/780,379, filed Mar. 9, 2006. All applications above are incorporated herein in their entirety.

### FIELD OF THE INVENTION

This invention provides for the use of compounds represented by the structure of the general formula (A):

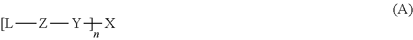

wherein L is a lipid or a phospholipid, Z is either nothing, ethanolamine, serine, inositol, choline, or glycerol, Y is either nothing or a spacer group ranging in length from 2 to 30 atoms, X is a physiologically acceptable monomer, dimer, oligomer, or polymer, wherein X is a glycosaminoglycan; and n is a number from 1 to 1000, wherein any bond between L, Z, Y and X is either an amide or an esteric bond for the treatment of a subject suffering from cystic fibrosis, reduction or delay in mortality associated with cystic fibrosis or amelioration of symptoms associated with cystic fibrosis.

### BACKGROUND OF THE INVENTION

Cystic fibrosis (CF) is a prominent genetic pulmonary disease that is inherited in an autosomal recessive manner and affects children and young adults. The clinical features of CF are dominated by involvement of the respiratory tract, where obstruction of the airways by copious amounts of unusually thick mucus and subsequent infections, especially with Pseudomonas species, predominate. There is also involvement of the gastrointestinal tract in most patients, including malabsorption and pancreatic insufficiency. The affected tissue in CF is the secretory epithelia, which mediates the transport of water, salt, and other solutes at an interface between the blood and a lumen. CF epithelial cells in the skin, lungs and digestive tract cannot properly transport chloride through their membranes, thereby altering water secretion and mucus production.

The defective gene in this disorder has been recently cloned and is known as CFTR (cystic fibrosis transmembrane conductance regulator). The CFTR gene product is a protein that functions as a regulated transport channel for chloride ions. Point mutations and deletions in the CFTR gene result in the expression of a defective chloride ion transport channel in epithelial cells, causing the subsequent deleterious symptoms of CF.

There are numerous manifestations of bronchopulmonary viral and microbial infections in individuals with CF. Because of a resurgence in antibiotic-resistant strains, many of these infections are a cause of great concern, for example, tuberculosis caused by drug resistant strains of Mycobacterium tuberculosis. Other species that cause diseases such as pneumonia also exhibit increasing drug resistance. Moreover, viral infections cannot be treated with antibiotics, and few satisfactory anti-viral medications are available. A secondary effect of the unusual mucosal environment of the CF lung is bronchopulmonary infection associated with chronic progressive lung disease and episodes of acute exacerbation. Colonization of the airways with Pseudomonas aeruginosa and cross-infection with Pseudomonas cepacia is a major cause of pulmonary deterioration in CF. Members of the Pseudomonas genus are well-known as opportunistic pathogens that have an innate resistance to most commonly used antibiotics. Accordingly, it would be a significant advance in the art to develop an alternative method of treating these microbial and viral bronchopulmonary infections.

Lipid-conjugates having a pharmacological activity of inhibiting the enzyme phospholipase A2 (PLA2, EC 3.1.1.4) are known in the prior art. Phospholipase A2 catalyzes the breakdown of phospholipids at the sn-2 position to produce a fatty acid and a lysophospholipid. The activity of this enzyme has been correlated with various cell functions, particularly with the production of lipid mediators such as eicosanoid production (prostaglandins, thromboxanes and leukotrienes), platelet activating factor and lysophospholipids. Since their inception, lipid-conjugates have been subjected to intensive laboratory investigation in order to obtain a wider scope of protection of cells and organisms from injurious agents and pathogenic processes.

### SUMMARY OF THE INVENTION

In one embodiment, the invention provides a method of treating a subject suffering from cystic fibrosis, reducing or delaying the mortality of a subject suffering from cystic fibrosis or ameliorating symptoms associated with cystic fibrosis, the method comprising the step of administering a compound represented by the structure of the general formula (A):

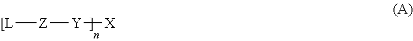

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein X is a glycosaminoglycan; and
n is a number from 1 to 1000;
wherein any bond between L, Z, Y and X is either an amide or an esteric bond to a subject afflicted with or suffering from symptoms of cystic fibrosis.

In one embodiment, the compound is represented by the structure of the general formula (I):

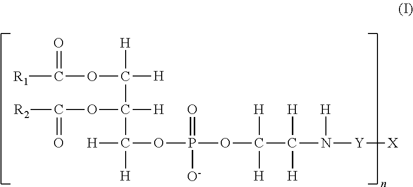

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is either a physiologically acceptable monomer, dimer, oligomer or a physiologically acceptable polymer, wherein X is a glycosaminoglycan; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

In one embodiment, the compound is represented by the structure of the general formula (II):

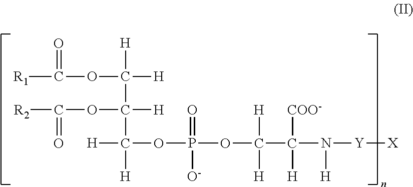

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer wherein x is a glycosaminoglycan; and
n is a number from 1 to 1000;
wherein if Y is nothing the phosphatidylserine is directly linked to X via an amide bond and if Y is a spacer, said spacer is directly linked to X via an amide or an esteric bond and to said phosphatidylserine via an amide bond.

In one embodiment, the compound is represented by the structure of the general formula (III):

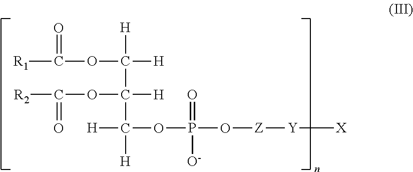

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;

Z is either nothing, inositol, choline, or glycerol;

Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phosphatidyl, Z, Y and X is either an amide or an esteric bond.

In one embodiment, the compound is represented by the structure of the general formula (IV):

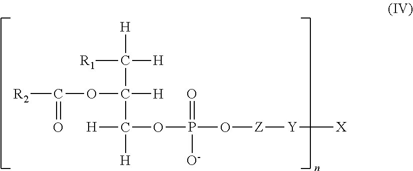

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In one embodiment, the compound is represented by the structure of the general formula (V):

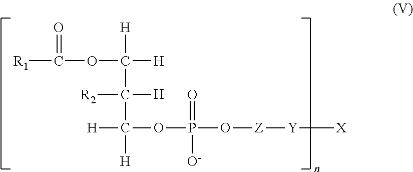

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In one embodiment, the compound is represented by the structure of the general formula (VI):

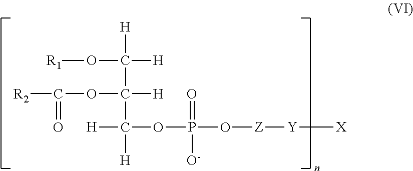

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In one embodiment, the compound is represented by the structure of the general formula (VII):

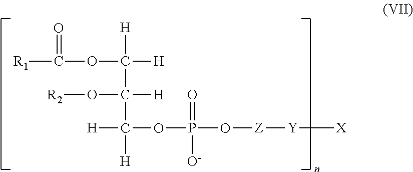

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In one embodiment, the compound is represented by the structure of the general formula (VIII):

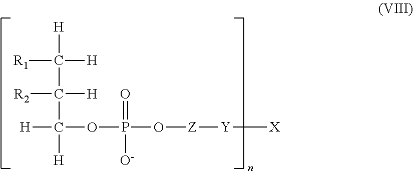

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In one embodiment, the compound is represented by the structure of the general formula (IX):

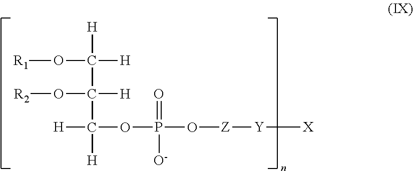

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In one embodiment, the compound is represented by the structure of the general formula (X):

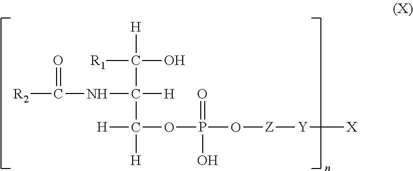

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the ceramide phosphoryl, Z, Y and X is either an amide or an esteric bond.

In one embodiment, the compound is represented by the structure of the general formula (XI):

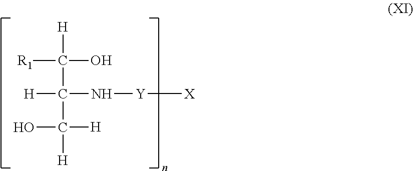

wherein
R₁ is a linear, saturated, mono-unsaturated, or poly-unsaturated, alkyl chain ranging in length from 2 to 30 carbon atoms;
Z is nothing;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000; wherein if Y is nothing the sphingosyl is directly linked to X via an amide bond and if
Y is a spacer, said spacer is directly linked to X and to said sphingosyl via an amide bond and to X via an amide or an esteric bond.

In one embodiment, the compound is represented by the structure of the general formula (XII):

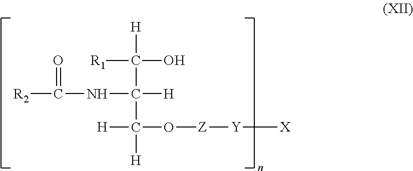

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the ceramide, Z, Y and X is either an amide or an esteric bond.

In one embodiment, the compound is represented by the structure of the general formula (XIII):

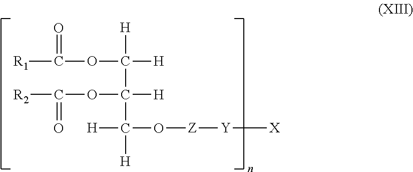

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the diglyceryl, Z, Y and X is either an amide or an esteric bond.

In one embodiment, the compound is represented by the structure of the general formula (XIV):

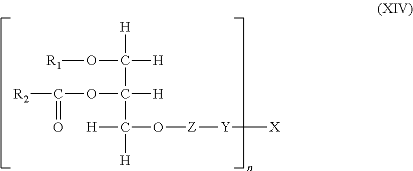

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the glycerolipid, Z, Y and X is either an amide or an esteric bond.

In one embodiment, the compound is represented by the structure of the general formula (XV):

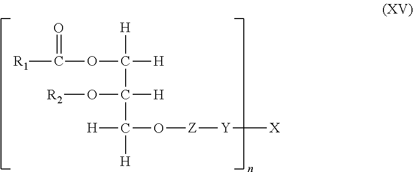

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the glycerolipid, Z, Y and X is either an amide or an esteric bond.

In one embodiment, the compound is represented by the structure of the general formula (XVI):

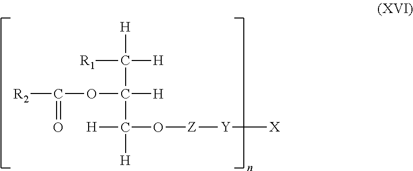

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between said lipid, Z, Y and X is either an amide or an esteric bond.

In one embodiment, the compound is represented by the structure of the general formula (XVII):

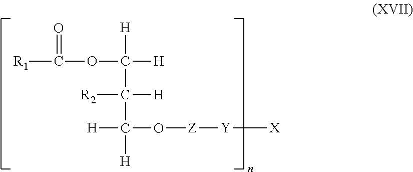

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In one embodiment, the compound is represented by the structure of the general formula (XVIII):

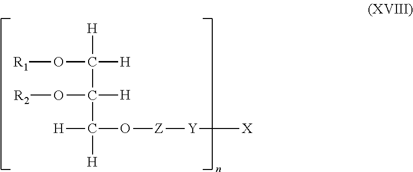

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In one embodiment, the compound is represented by the structure of the general formula (XIX):

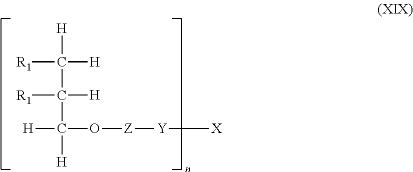

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In one embodiment, the compound is represented by the structure of the general formula (XX):

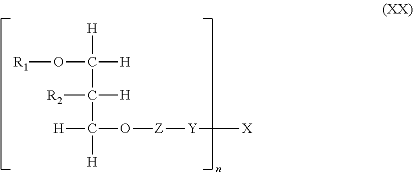

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In one embodiment, the compound is represented by the structure of the general formula (XXI):

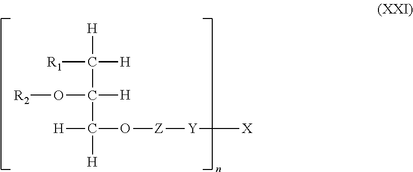

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In one embodiment, the compound comprises a glycosaminoglycan, which is hyaluronic acid, heparin, heparan sulfate, chondrotin sulfate, keratin, keratan sulfate, dermatan sulfate or a derivative thereof.

In one embodiment, the compound comprises a glycosaminoglycan, which comprises di- and trisaccharide unit monomers of glycosaminoglycans.

In one embodiment, the compound comprises a chondroitin sulfate, which is chondroitin-6-sulfate, chondroitin-4-sulfate or a derivative thereof.

In one embodiment, the compound comprises a glycosaminoglycan comprising intact sugar rings.

In one embodiment, the compound comprises dipalmitoyl phosphatidylethanolamine and heparin.

In one embodiment, the compound comprises dipalmitoyl phosphatidylethanolamine and chondroitin sulfate.

In one embodiment, the compound comprises dipalmitoyl phosphatidylethanolamine and hyaluronic acid.

In one embodiment, the compound comprises dipalmitoyl phosphatidylethanolamine and carboxymethylcellulose.

In one embodiment, the compound comprises dimyristoyl phosphatidylethanolamine and hyaluronic acid.

In one embodiment, the method diminishes or abrogates a deleterious inflammatory response in said subject.

In one embodiment, the method prevents, treats, reduces the incidence of, reduces the severity of, delays the onset of, or diminishes the pathogenesis of an infection is said subject.

In one embodiment, the invention provides a method for decreasing expression of proinflammatory chemokines, cytokines, or a combination thereof comprising the step of administering a compound represented by the structure of the general formula (A) as described hereinabove.

In one embodiment, the invention provides a method of activating NF-κB, IL-6, IL-8, or a combination thereof in human airway epithelial cell lines comprising the step of administering to a subject a compound represented by the structure of the general formula (A) as described hereinabove.

### BRIEF DESCRIPTION OF DRAWINGS

FIG. 1A: Effect of Lipid-conjugates on cytokine levels in Pseudomonas-infected and uninfected 16HBE+CFTR sense (non CF-like) and 16HBE+CFTR antisense (CF-like) bronchial epithelial cells.

FIG. 1B: Effect of Lipid-conjugates on cytokine levels in Pseudomonas-infected and uninfected C38 (non CF-like) and IB3 (CF-like) bronchial epithelial cells.

### DETAILED DESCRIPTION OF THE INVENTION

In one embodiment, this invention provides a method for treating a subject suffering from cystic fibrosis, reducing or delaying the mortality of a subject suffering from cystic fibrosis or ameliorating symptoms associated with cystic fibrosis via administration of a compound comprising a lipid or a phospholipid bonded, directly or via a spacer group, to a physiologically acceptable monomer, dimer, oligomer, or polymer.

In one embodiment, this invention provides for the use of a number of compounds, for application in treating, preventing, suppressing, etc., cystic fibrosis, as further described hereinbelow.

### Compounds

In one embodiment, reference to a compound for use in a method of the present invention refers to one comprising a lipid or phospholipid moiety bound to a physiologically acceptable monomer, dimer, oligomer, or polymer. In one embodiment, the compounds for use in the present invention are referred to as “Lipid-conjugates.” In one embodiment, compounds for use in the present invention are described by the general formula:

[phosphatidylethanolamine-Y]n-X

[phosphatidylserine-Y]n-X

[phosphatidylcholine-Y]n-X

[phosphatidylinositol-Y]n-X

[phosphatidylglycerol-Y]n-X

[phosphatidic acid-Y]n-X

[lyso-phospholipid-Y]n-X

[diacyl-glycerol-Y]n-X

[monoacyl-glycerol-Y]n-X

[sphingomyelin-Y]n-X

[sphingosine-Y]n-X

[ceramide-Y]n-X

wherein
Y is either nothing or a spacer group ranging in length from 2 to 30 atoms; and
X is a physiologically acceptable monomer, dimer, oligomer or polymer; and
n is the number of lipid molecules bound to a molecule of X, wherein n is a number from 1 to 1000.

In one embodiment, the invention provides low-molecular weight Lipid-conjugates, which possess pharmacological activity, which are characterized by the general formula described hereinabove.

In one embodiment of the invention, the physiologically acceptable monomer is salicylate. In another embodiment, the physiologically acceptable monomer is salicylic acid. In another embodiment, the physiologically acceptable monomer is acetyl salicylic acid. In another embodiment, the physiologically acceptable monomer is aspirin. In another embodiment, the physiologically acceptable monomer is a monosaccharide. In another embodiment, the physiologically acceptable monomer is lactobionic acid. In another embodiment, the physiologically acceptable monomer is glucoronic acid. In another embodiment, the physiologically acceptable monomer is maltose. In another embodiment, the physiologically acceptable monomer is an amino acid. In another embodiment, the physiologically acceptable monomer is glycine. In another embodiment, the physiologically acceptable monomer is a carboxylic acid. In another embodiment, the physiologically acceptable monomer is an acetic acid. In another embodiment, the physiologically acceptable monomer is a butyric acid. In another embodiment, the physiologically acceptable monomer is a dicarboxylic acid. In another embodiment, the physiologically acceptable monomer is a fatty acid. In another embodiment, the physiologically acceptable monomer is a dicarboxylic fatty acid. In another embodiment, the physiologically acceptable monomer is a glutaric acid. In another embodiment, the physiologically acceptable monomer is succinic acid. In another embodiment, the physiologically acceptable monomer is dodecanoic acid. In another embodiment, the physiologically acceptable monomer is didodecanoic acid. In another embodiment, the physiologically acceptable monomer is bile acid. In another embodiment, the physiologically acceptable monomer is cholic acid. In another embodiment, the physiologically acceptable monomer is cholesterylhemisuccinate.

In one embodiment of the invention, the physiologically acceptable dimer or oligomer is a dipeptide. In another embodiment, the physiologically acceptable dimer or oligomer is a disaccharide. In another embodiment, the physiologically acceptable dimer or oligomer is a trisaccharide. In another embodiment, the physiologically acceptable dimer or oligomer is an oligosaccharide. In another embodiment, the physiologically acceptable dimer or oligomer is an oligopeptide. In another embodiment, the physiologically acceptable dimer or oligomer is a glycoprotein mixture. In another embodiment, the physiologically acceptable dimer or oligomer is a di- or trisaccharide monomer unit of a polysaccharide. In another embodiment, the physiologically acceptable dimer or oligomer is a di- or trisaccharide monomer unit of a polypyranose. In another embodiment, the physiologically acceptable dimer or oligomer is a di- or trisaccharide monomer unit of a glycosaminogylcan. In another embodiment, the physiologically acceptable dimer or oligomer is a di- or trisaccharide monomer unit of a hyaluronic acid. In another embodiment, the physiologically acceptable dimer or oligomer is a di- or trisaccharide monomer unit of a heparin. In another embodiment, the physiologically acceptable dimer or oligomer is a di- or trisaccharide monomer unit of a heparan sulfate. In another embodiment, the physiologically acceptable dimer or oligomer is a di- or trisaccharide monomer unit of a keratin. In another embodiment, the physiologically acceptable dimer or oligomer is a di- or trisaccharide monomer unit of a keratan sulfate. In another embodiment, the physiologically acceptable dimer or oligomer is a di- or trisaccharide monomer unit of a chondroitin. In another embodiment, the chondroitin is chondoitin sulfate. In another embodiment, the chondroitin is chondoitin-4-sulfate. In another embodiment, the chondroitin is chondoitin-6-sulfate. In another embodiment, the physiologically acceptable dimer or oligomer is a di- or trisaccharide monomer unit of a dermatin. In another embodiment, the physiologically acceptable dimer or oligomer is a di- or trisaccharide monomer unit of a dermatan sulfate. In another embodiment, the physiologically acceptable dimer or oligomer is dextran. In another embodiment, the physiologically acceptable dimer or oligomer is polygeline (‘Haemaccel’). In another embodiment, the physiologically acceptable dimer or oligomer is alginate, In another embodiment, the physiologically acceptable dimer or oligomer is hydroxyethyl starch (Hetastarch). In another embodiment, the physiologically acceptable dimer or oligomer is ethylene glycol. In another embodiment, the physiologically acceptable dimer or oligomer is carboxylated ethylene glycol.

In one embodiment, the physiologically acceptable polymer is a polysaccharide. In another embodiment, the physiologically acceptable polymer is a homo-polysaccharide. In another embodiment, the physiologically acceptable polymer is a hetero-polysaccharide. In another embodiment, the physiologically acceptable polymer is a polypyranose. In another embodiment of the invention, the physiologically acceptable polymer is a glycosaminoglycan. In another embodiment, the physiologically acceptable polymer is hyaluronic acid. In another embodiment, the physiologically acceptable polymer is heparin. In another embodiment, the physiologically acceptable polymer is heparan sulfate. In another embodiment, the physiologically acceptable polymer is chondroitin. In another embodiment, the chondroitin is chondoitin-4-sulfate. In another embodiment, the chondroitin is chondoitin-6-sulfate. In another embodiment, the physiologically acceptable polymer is keratin. In another embodiment, the physiologically acceptable polymer is keratan sulfate. In another embodiment, the physiologically acceptable polymer is dermatin. In another embodiment, the physiologically acceptable polymer is dermatan sulfate. In another embodiment, the physiologically acceptable polymer is carboxymethylcellulose. In another embodiment, the physiologically acceptable polymer is dextran. In another embodiment, the physiologically acceptable polymer is polygeline (‘Haemaccel’). In another embodiment, the physiologically acceptable polymer is alginate. In another embodiment, the physiologically acceptable polymer is hydroxyethyl starch (‘Hetastarch’). In another embodiment, the physiologically acceptable polymer is polyethylene glycol. In another embodiment, the physiologically acceptable polymer is polycarboxylated polyethylene glycol. In another embodiment, the physiologically acceptable polymer is a peptide. In another embodiment, the physiologically acceptable polymer is an oligopeptide. In another embodiment, the physiologically acceptable polymer is a polyglycan. In another embodiment, the physiologically acceptable polymer is a protein. In another embodiment, the physiologically acceptable polymer is a glycoprotein mixture.

In one embodiment, examples of polymers which can be employed as the conjugated moiety for producing Lipid-conjugates for use in the methods of this invention may be physiologically acceptable polymers, including water-dispersible or -soluble polymers of various molecular weights and diverse chemical types, mainly natural and synthetic polymers, such as glycosaminoglycans as described hereinabove, plasma expanders, including polygeline (“Haemaccel”, degraded gelatin polypeptide cross-linked via urea bridges, produced by “Behring”), “hydroxyethylstarch” (Hetastarch, HES) and extrans, food and drug additives, soluble cellulose derivatives (e.g., methylcellulose, carboxymethylcellulose), polyaminoacids, hydrocarbon polymers (e.g., polyethylene), polystyrenes, polyesters, polyamides, polyethylene oxides (e.g. polyethyleneglycols, polycarboxyethyleneglycols, polycarboxylated polyethyleneglycols), polyvinnylpyrrolidones, polysaccharides, polypyranoses, alginates, assimilable gums (e.g., xanthan gum), peptides, injectable blood proteins (e.g., serum albumin), cyclodextrin, and derivatives thereof.

In one embodiment of the invention, the lipid or phospholipid moiety is phosphatidic acid. In another embodiment, lipid or phospholipid moiety is an acyl glycerol. In another embodiment, lipid or phospholipid moiety is monoacylglycerol. In another embodiment, lipid or phospholipid moiety is diacylglycerol. In another embodiment, lipid or phospholipid moiety is triacylglycerol. In another embodiment, lipid or phospholipid moiety is sphingosine. In another embodiment, lipid or phospholipid moiety is sphingomyelin. In another embodiment, lipid or phospholipid moiety is ceramide. In another embodiment, lipid or phospholipid moiety is phosphatidylethanolamine. In another embodiment, lipid or phospholipid moiety is phosphatidylserine. In another embodiment, lipid or phospholipid moiety is phosphatidylcholine. In another embodiment, lipid or phospholipid moiety is phosphatidylinositol. In another embodiment, lipid or phospholipid moiety is phosphatidylglycerol. In another embodiment, lipid or phospholipid moiety is an ether or alkyl phospholipid derivative thereof.

In one embodiment, the set of compounds comprising phosphatidylethanolamine covalently bound to a physiologically acceptable monomer, dimmer, oligomer, or polymer, is referred to herein as the PE-conjugates. In one embodiment, the phosphatidylethanolamine moiety is dipalmitoyl phosphatidylethanolamine. In another embodiment, the phosphatidylethanolamine moiety is dimyristoyl phosphatidylethanolamine. In another embodiment, related derivatives, in which either phosphatidylserine, phosphatidylcholine, phosphatidylinositol, phosphatidic acid or phosphatidylglycerol are employed in lieu of phosphatidylethanolamine as the lipid moiety provide equivalent therapeutic results, based upon the biological experiments described below for the Lipid-conjugates and the structural similarities shared by these compounds.

As defined by the structural formulae provided herein for the Lipid-conjugates, these compounds may contain between one to one thousand lipid moieties bound to a single physiologically acceptable polymer molecule. In one embodiment of this invention, n is a number from 1 to 1000. In another embodiment, n is a number from 1 to 500. In another embodiment, n is a number from 1 to 100. In another embodiment, n is a number from 2 to 1000. In another embodiment, n is a number from 2 to 100. In another embodiment, n is a number from 2 to 200. In another embodiment, n is a number from 3 to 300. In another embodiment, n is a number from 10 to 400. In another embodiment, n is a number from 50 to 500. In another embodiment, n is a number from 100 to 300. In another embodiment, n is a number from 300 to 500. In another embodiment, n is a number from 500 to 800. In another embodiment, n is a number from 500 to 1000.

In one embodiment of the invention, when the conjugated moiety is a polymer, the ratio of lipid moieties covalently bound may range from one to one thousand lipid residues per polymer molecule, depending upon the nature of the polymer and the reaction conditions employed. For example, the relative quantities of the starting materials, or the extent of the reaction time, may be modified in order to obtain Lipid-conjugate products with either high or low ratios of lipid residues per polymer, as desired.

In the methods, according to embodiments of the invention, the Lipid-conjugates administered to a subject are comprised of at least one lipid moiety covalently bound through an atom of the polar head group to a monomeric or polymeric moiety (referred to herein as the conjugated moiety) of either low or high molecular weight. In one embodiment, the conjugated moiety is conjugated to the lipid, phospholipid, or spacer via an ester bond. In another embodiment, the conjugated moiety is conjugated to the lipid, phospholipid, or spacer via an amide bond.

When desired, an optional bridging moiety can be used to link the Lipid-conjugates moiety to the monomer or polymeric moiety. The composition of some phospholipid-conjugates of high molecular weight, and associated analogues, are the subject of U.S. Pat. No. 5,064,817, which is incorporated herein in its entirety by reference.

In one embodiment, the term “moiety” means a chemical entity otherwise corresponding to a chemical compound, which has a valence satisfied by a covalent bond.

In some cases, according to embodiments of the invention, the monomer or polymer chosen for preparation of the Lipid-conjugate may in itself have select biological properties. For example, both heparin and hyaluronic acid are materials with known physiological functions. In the present invention, however, the Lipid-conjugates formed from these substances as starting materials display a new and wider set of pharmaceutical activities than would be predicted from administration of either heparin or hyaluronic acid which have not been bound by covalent linkage to a phospholipid. It can be shown, by standard comparative experiments that phosphatidylethanolamine (PE) linked to hyaluronic acid (Compound XXII), to heparin (Compound XXIV), to chondroitin sulfate A (Compound XXV), to carboxymethylcellulose (Compound XXVI), to Polygeline (haemaccel) (Compound XXVII), or to hydroxyethylstarch (Compound XXVIII), are far superior in terms of potency and range of useful pharmaceutical activity to the free conjugates (the polymers above and the like). In fact, these latter substances are, in general, not considered useful in methods for treatment of cystic fibrosis. Thus, the combination of a phospholipid such as phosphatidylethanolamine, or related phospholipids which differ with regard to the polar head group, such as phosphatidylserine (PS), phosphatidylcholine (PC), phosphatidylinositol (PI), and phosphatidylglycerol (PG), results in the formation of a compound which has novel pharmacological properties when compared to the starting materials alone. In the cases described herein, the diversity of biological activities and the effectiveness in disease exhibited by the compounds far exceed the properties anticipated by use of the starting materials themselves, when administered alone or in combination.

The biologically active Lipid-conjugates described herein can have a wide range of molecular weights, e.g., above 50,000 (up to a few hundred thousands) when it is desirable to retain the Lipid conjugate in the vascular system and below 50,000 when targeting to extravascular systems is desirable. The sole limitation on the molecular weight and the chemical structure of the conjugated moiety is that it does not result in a Lipid-conjugate devoid of the desired biological activity, or lead to chemical or physiological instability to the extent that the Lipid-conjugate is rendered useless as a drug in the method of use described herein.

In one embodiment, the compound for use in the present invention is represented by the structure of the general formula (A):

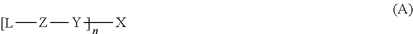

wherein
L is a lipid or a phospholipid;
Z is either nothing, ethanolamine, serine, inositol, choline, phosphate, or glycerol;
Y is either nothing or a spacer group ranging in length from 2 to 30 atoms;
X is a physiologically acceptable monomer, dimer, oligomer, or polymer; and
n is a number from 1 to 1000;
wherein any bond between L, Z, Y and X is either an amide or an esteric bond.

In one embodiment, L is phosphatidyl, Z is ethanolamine, wherein L and Z are chemically bonded resulting in phosphatidylethanolamine, Y is nothing, and X is carboxymethylcellulose. In another embodiment, L is phosphatidyl, Z is ethanolamine, wherein L and Z are chemically bonded resulting in phosphatidylethanolamine, Y is nothing, and X is a glycosaminoglycan. In one embodiment, the phosphatidylethanolamine moiety is dipalmitoyl phosphatidylethanolamine. In another embodiment, the phosphatidylethanolamine moiety is dimyristoyl phosphatidylethanolamine.

In another embodiment, the compound for use in the present invention is represented by the structure of the general formula (I):

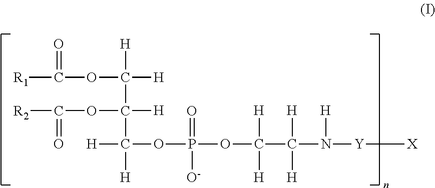

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms; and
X is either a physiologically acceptable monomer, dimer, oligomer or a physiologically acceptable polymer; and
n is a number from 1 to 1,000;
wherein if Y is nothing the phosphatidylethanolamine is directly linked to X via an amide bond and if Y is a spacer, the spacer is directly linked to X via an amide or an esteric bond and to the phosphatidylethanolamine via an amide bond.

In one embodiment, compounds for use in the methods of the invention comprise one of the following as the conjugated moiety X: acetate, butyrate, glutarate, succinate, dodecanoate, didodecanoate, maltose, lactobionic acid, dextran, alginate, aspirin, cholate, cholesterylhemisuccinate, carboxymethyl-cellulose, heparin, hyaluronic acid, chondroitin sulfate, polygeline (haemaccel), polyethyleneglycol, polycarboxylated polyethylene glycol, a glycosaminoglycan, a polysaccharide, a hetero-polysaccharide, a homo-polysaccharide, or a polypyranose. The polymers used as starting material to prepare the PE-conjugates may vary in molecular weight from 1 to 2,000 kDa.

Examples of phosphatidylethanolamine (PE) moieties are analogues of the phospholipid in which the chain length of the two fatty acid groups attached to the glycerol backbone of the phospholipid varies from 2-30 carbon atoms length, and in which these fatty acids chains contain saturated and/or unsaturated carbon atoms. In lieu of fatty acid chains, alkyl chains attached directly or via an ether linkage to the glycerol backbone of the phospholipid are included as analogues of PE. In one embodiment, the PE moiety is dipalmitoyl-phosphatidyl-ethanolamine. In another embodiment, the PE moiety is dimyristoyl-phosphatidyl-ethanolamine.

Phosphatidyl-ethanolamine and its analogues may be from various sources, including natural, synthetic, and semisynthetic derivatives and their isomers.

Phospholipids which can be employed in lieu of the PE moiety are N-methyl-PE derivatives and their analogues, linked through the amino group of the N-methyl-PE by a covalent bond; N,N-dimethyl-PE derivatives and their analogues linked through the amino group of the N,N-dimethyl-PE by a covalent bond, phosphatidylserine (PS) and its analogues, such as palmitoyl-stearoyl-PS, natural PS from various sources, semisynthetic PSs, synthetic, natural and artifactual PSs and their isomers. Other phospholipids useful as conjugated moieties in this invention are phosphatidylcholine (PC), phosphatidylinositol (PI), phosphatidic acid and phosphoatidylglycerol (PG), as well as derivatives thereof comprising either phospholipids, lysophospholipids, phosphatidic acid, sphingomyelins, lysosphingomyelins, ceramide, and sphingosine.

For PE-conjugates and PS-conjugates, the phospholipid is linked to the conjugated monomer or polymer moiety through the nitrogen atom of the phospholipid polar head group, either directly or via a spacer group. For PC, PI, and PG conjugates, the phospholipid is linked to the conjugated monomer or polymer moiety through either the nitrogen or one of the oxygen atoms of the polar head group, either directly or via a spacer group.

In another embodiment, the compound for use in the present invention is represented by the structure of the general formula (II):

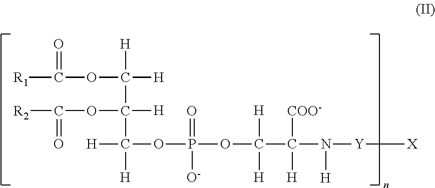

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein if Y is nothing, the phosphatidylserine is directly linked to X via an amide bond and if Y is a spacer, the spacer is directly linked to X via an amide or an esteric bond and to the phosphatidylserine via an amide bond.

In one embodiment, the phosphatidylserine may be bonded to Y, or to X if Y is nothing, via the COO⁻ moiety of the phosphatidylserine.

In another embodiment, the compound for use in the present invention is represented by the structure of the general formula (III):

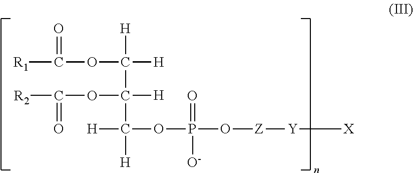

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phosphatidyl, Z, Y and X is either an amide or an esteric bond.

In another embodiment, the compound for use in the present invention is represented by the structure of the general formula (IV):

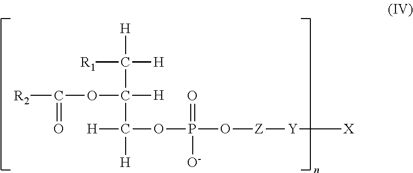

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (V):

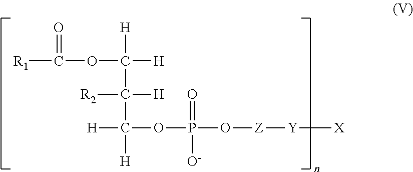

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (VI):

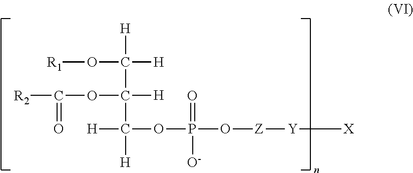

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (VII):

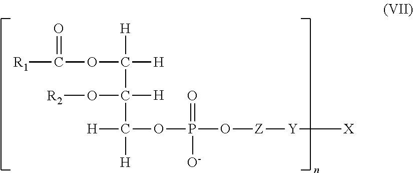

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In one embodiment of the invention, phosphatidylcholine (PC), phosphatidylinositol (PI), phosphatidic acid (PA), wherein Z is nothing, and phosphatidylglycerol (PG) conjugates are herein defined as compounds of the general formula (III).

In another embodiment, the compound for use in the present invention is represented by the structure of the general formula (VIII):

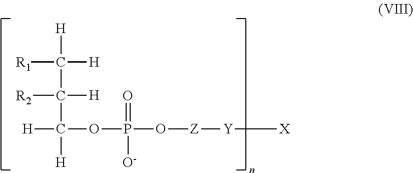

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (IX):

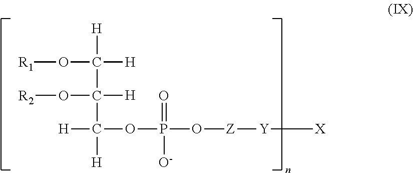

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (IXa):

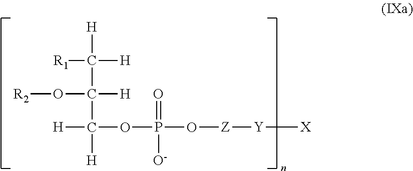

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (IXb):

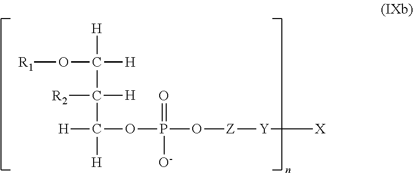

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the phospholipid, Z, Y and X is either an amide or an esteric bond.

In another embodiment, the compound for use in the present invention is represented by the structure of the general formula (X):

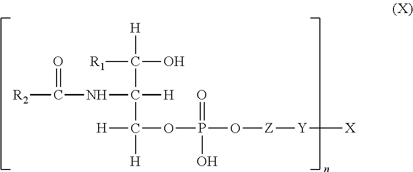

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer wherein X is a glycosaminoglycan; and
n is a number from 1 to 1000;
wherein any bond between the ceramide phosphoryl, Z, Y and X is either an amide or an esteric bond.

In another embodiment, the compound for use in the present invention is represented by the structure of the general formula (XI):

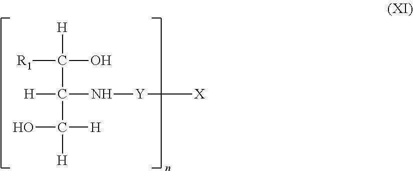

wherein
R₁ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein if Y is nothing the sphingosyl is directly linked to X via an amide bond and if
Y is a spacer, the spacer is directly linked to X and to the sphingosyl via an amide bond and to X via an amide or an esteric bond.

In another embodiment, the compound for use in the present invention is represented by the structure of the general formula (XII):

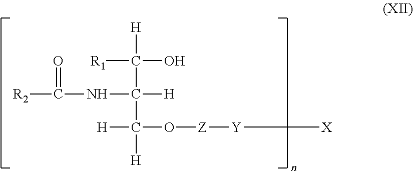

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the ceramide, Z, Y and X is either an amide or an esteric bond.

In another embodiment, the compound for use in the present invention is represented by the structure of the general formula (XIII):

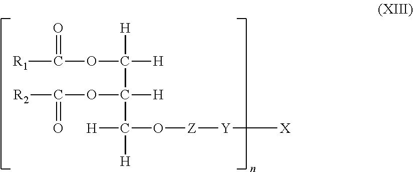

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the diglyceryl, Z, Y and X is either an amide or an esteric bond.

In another embodiment, the compound for use in the present invention is represented by the structure of the general formula (XIV):

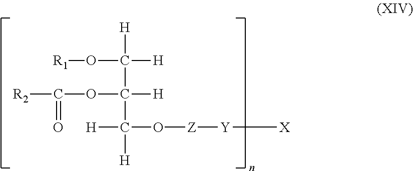

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the glycerolipid, Z, Y and X is either an amide or an esteric bond.

In another embodiment, the compound for use in the present invention is represented by the structure of the general formula (XV):

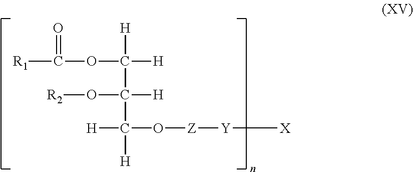

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the glycerolipid, Z, Y and X is either an amide or an esteric bond.

In another embodiment, the compound for use in the present invention is represented by the structure of the general formula (XVI):

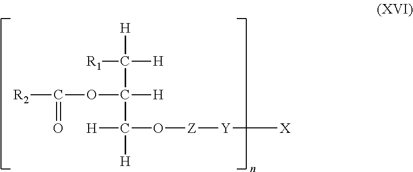

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the lipid, Z, Y and X is either an amide or an esteric bond.

In another embodiment, the compound for use in the present invention is represented by the structure of the general formula (XVII):

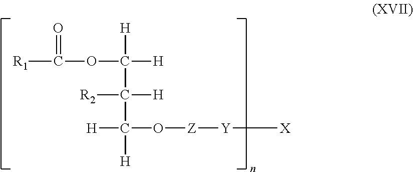

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the lipid, Z, Y and X is either an amide or an esteric bond.

In another embodiment, the compound for use in the present invention is represented by the structure of the general formula (XVIII):

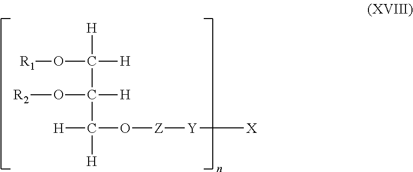

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the lipid, Z, Y and X is either an amide or an esteric bond.

In another embodiment, the compound for use in the present invention is represented by the structure of the general formula (XIX):

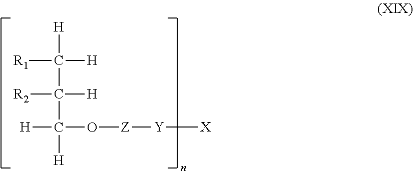

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the lipid, Z, Y and X is either an amide or an esteric bond.

In another embodiment, the compound for use in the present invention is represented by the structure of the general formula (XX):

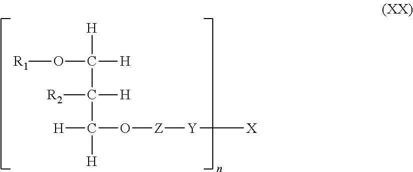

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the lipid, Z, Y and X is either an amide or an esteric bond.

In another embodiment, the compound for use in the present invention is represented by the structure of the general formula (XXI):

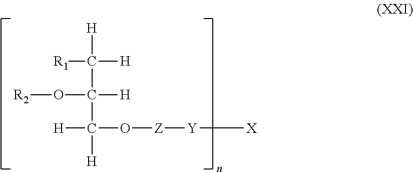

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer wherein X is a glycosaminoglycan; and
n is a number from 1 to 1000;
wherein any bond between the lipid, Z, Y and X is either an amide or an esteric bond.

For any or all of the compounds represented by the structures of the general formulae (A), (I), (II), (III), (IV), (V), (VI), (VII), (VIII), (IX), (IXa), (IXb), (X), (XI), (XII), (XIII), (XIV), (XV), (XVI), (XVII), (XVIII), (XIX), (XX), (XXI), and (XXII) hereinabove: In one embodiment, X is a glycosaminoglycan. According to this aspect and in one embodiment, the glycosaminoglycan may be, inter alia, hyaluronic acid, heparin, heparan sulfate, chondroitin sulfate, keratin, keratan sulfate, dermatan sulfate or a derivative thereof. In one embodiment, the chondroitin sulfate may be, inter alia, chondroitin-6-sulfate, chondroitin-4-sulfate or a derivative thereof. In another embodiment, X is not a glycosaminoglycan. In another embodiment, X is a polysaccharide, which in one embodiment is a hetero-polysaccharide, and in another embodiment, is a homo-polysaccharide. In another embodiment, X is a polypyranose.

In another embodiment, the glycosaminoglycan is a polymer of disaccharide units. In another embodiment, the number of the disaccharide units in the polymer is m. In another embodiment, m is a number from 2-10,000. In another embodiment, m is a number from 2-500. In another embodiment, m is a number from 2-1000. In another embodiment, m is a number from 50-500. In another embodiment, m is a number from 2-2000. In another embodiment, m is a number from 500-2000. In another embodiment, m is a number from 1000-2000. In another embodiment, m is a number from 2000-5000. In another embodiment, m is a number from 3000-7000. In another embodiment, m is a number from 5000-10,000. In another embodiment, a disaccharide unit of a glycosaminoglycan may be bound to one lipid or phospholipid moiety. In another embodiment, each disaccharide unit of the glycosaminoglycan may be bound to zero or one lipid or phospholipid moieties. In another embodiment, the lipid or phospholipid moieties are bound to the —COOH group of the disaccharide unit. In another embodiment, the bond between the lipid or phospholipid moiety and the disaccharide unit is an amide bond.

In one embodiment of the invention, Y is nothing. Non-limiting examples of suitable divalent groups forming the optional bridging group (which in one embodiment, is referred to as a spacer) Y, according to embodiments of the invention, are straight or branched chain alkylene, e.g., of 2 or more, preferably 4 to 30 carbon atoms, —CO-alkylene-CO, —NH-alkylene-NH—, —CO-alkylene-NH—, —NH-alkylene-NH, CO-alkylene-NH—, an amino acid, cycloalkylene, wherein alkylene in each instance, is straight or branched chain and contains 2 or more, preferably 2 to 30 atoms in the chain, —(—O—CH(CH₃)CH₂)ₓ— wherein x is an integer of 1 or more.

In one embodiment of the invention, the sugar rings of the glycosaminoglycan are intact. In another embodiment, intact refers to closed. In another embodiment, intact refers to natural. In another embodiment, intact refers to unbroken.

In one embodiment of the invention, the structure of the lipid or phospholipid in any compound according to the invention is intact. In another embodiment, the natural structure of the lipid or phospholipids in any compound according to the invention is maintained.

In one embodiment, the compounds for use in the present invention are biodegradable.

In one embodiment, the compound according to the invention is phosphatidylethanolamine bound to aspirin. In one embodiment, the compound according to the invention is phosphatidylethanolamine bound to glutarate.

In some embodiments, the compounds for use are as listed in Table 1 below.

TABLE 1

| Phospholipid | | | |
|---|---|---|---|
| Compound | Spacer | Polymer (m.w.) | |
| PE | None | Hyaluronic acid | XXII |
| | | (2-2000 kDa) | |
| Dimyristoyl- | None | Hyaluronic acid | XXIII |
| PE | | | |
| PE | None | Heparin | XXIV |
| | | (0.5-110 kDa) | |
| PE | None | Chondroitin sulfate A | XXV |
| PE | None | Carboxymethylcellulose | XXVI |
| | | (20-500 kDa) | |
| PE | Dicarboxylic | Polygeline (haemaccel) | XXVII |
| | acid + Diamine | (4-40 kDa) | |
| PE | None | Hydroxyethylstarch | XXVIII |
| PE | Dicarboxylic | Dextran | XXIX |
| | acid + Diamine | (1-2,000 kDa) | |
| PE | None | Aspirin | XXX |
| PE | Carboxyl amino | Hyaluronic acid | XXXI |
| | group | (2-2000 kDa) | |
| PE | Dicarboxyl group | Hyaluronic acid | XXXII |
| | | (2-2000 kDa) | |
| PE | Dipalmitoic acid | Hyaluronic acid | XXXIII |
| | | (2-2000 kDa) | |
| PE | Carboxyl amino | Heparin | XXXIV |
| | group | (0.5-110 kDa) | |
| PE | Dicarboxyl group | Heparin | XXXV |
| | | (0.5-110 kDa) | |
| PE | Carboxyl amino | Chondroitin sulfate A | XXXVI |
| | group | | |
| PE | Dicarboxyl group | Chondroitin sulfate A | XXXVII |
| PE | Carboxyl amino | Carboxymethylcellulose | XXXVIII |
| | group | (20-500 kDa) | |
| PE | Dicarboxyl group | Carboxymethylcellulose | XXXIX |
| | | (20-500 kDa) | |
| PE | None | Polygeline (haemaccel) | XL |
| | | (4-40 kDa) | |
| PE | Carboxyl amino | Polygeline (haemaccel) | XLI |
| | group | (4-40 kDa) | |
| PE | Dicarboxyl group | Polygeline (haemaccel) | XLII |
| | | (4-40 kDa) | |
| PE | Carboxyl amino | Hydroxyethylstarch | XLIII |
| | group | | |
| PE | Dicarboxyl group | Hydroxyethylstarch | XLIV |
| PE | None | Dextran | XLV |
| | | (1-2,000 kDa) | |
| PE | Carboxyl amino | Dextran | XLVI |
| | group | (1-2,000 kDa) | |
| PE | Dicarboxyl group | Dextran | XLVII |
| | | (1-2,000 kDa) | |
| PE | Carboxyl amino | Aspirin | XLVIII |
| | group | | |
| PE | Dicarboxyl group | Aspirin | XLIX |
| PE | None | Albumin | L |
| PE | None | Alginate | LI |
| | | (2-2000 kDa) | |
| PE | None | Polyaminoacid | LII |
| PE | None | Polyethylene glycol | LIII |
| PE | None | Lactobionic acid | LIV |
| PE | None | Acetylsalicylate | LV |
| PE | None | Cholesteryl- | LVI |
| | | hemmisuccinate | |
| PE | None | Maltose | LVII |
| PE | None | Cholic acid | LVIII |
| PE | None | Chondroitin sulfates | LIX |
| PE | None | Polycarboxylated | LX |
| | | polyethylene glycol | |
| Dipalmitoyl- | None | Hyaluronic acid | LXI |
| PE | | | |
| Dipalmitoyl- | None | Heparin | LXII |
| PE | | | |
| Dipalmitoyl- | None | Chondroitin sulfate A | LXIII |
| PE | | | |
| Dipalmitoyl- | None | Carboxymethylcellulose | LXIV |
| PE | | | |
| Dipalmitoyl- | None | Polygeline (haemaccel) | LXV |
| PE | | | |
| Dipalmitoyl- | None | Hydroxyethylstarch | LXVI |
| PE | | | |
| Dipalmitoyl- | None | Dextran | LXVII |
| PE | | | |
| Dipalmitoyl- | None | Aspirin | LXVIII |
| PE | | | |
| Dimyristoyl- | None | Heparin | LXVIX |
| PE | | | |
| Dimyristoyl- | None | Chondroitin sulfate A | LXX |
| PE | | | |
| Dimyristoyl- | None | Carboxymethylcellulose | LXXI |
| PE | | | |
| Dimyristoyl- | None | Polygeline (haemaccel) | LXXII |
| PE | | | |
| Dimyristoyl- | None | Hydroxyethylstarch | LXXIII |
| PE | | | |
| Dimyristoyl- | None | Dextran | LXXIV |
| PE | | | |
| Dimyristoyl- | None | Aspirin | LXXV |
| PE | | | |
| PS | None | Hyaluronic acid | LXXVI |
| PS | None | Heparin | LXXVII |
| PS | None | Polygeline (haemaccel) | LXXVIII |
| PC | None | Hyaluronic acid | LXXIX |
| PC | None | Heparin | LXXX |
| PC | None | Polygeline (haemaccel) | LXXXI |
| PI | None | Hyaluronic acid | LXXXII |
| PI | None | Heparin | LXXXIII |
| PI | None | Polygeline (haemaccel) | LXXXIV |
| PG | None | Hyaluronic acid | LXXXV |
| PG | None | Heparin | LXXXVI |
| PG | None | Polygeline (haemaccel) | LXXXVII |
| PE | None | Glutaryl | LXXXVIII |

In one embodiment of the invention, the compounds for use in the present invention are any one or more of Compounds I-LXXXVIII. In another embodiment, the compounds for use in the present invention are Compound XXII, Compound XXIII, Compound XXIV, Compound XXV, Compound XXVI, Compound XXVII, Compound XXVIII, Compound XXIX, Compound XXX, or pharmaceutically acceptable salts thereof, in combination with a physiologically acceptable carrier or solvent. According to embodiments of the invention, these polymers, when chosen as the conjugated moiety, may vary in molecular weights from 200 to 2,000,000 Daltons. In one embodiment of the invention, the molecular weight of the polymer as referred to herein is from 200 to 1000 Daltons. In another embodiment, the molecular weight of the polymer as referred to herein is from 200 to 1000 Daltons. In another embodiment, the molecular weight of the polymer as referred to herein is from 1000 to 5000 Daltons. In another embodiment, the molecular weight of the polymer as referred to herein is from 5000 to 10,000 Daltons. In another embodiment, the molecular weight of the polymer as referred to herein is from 10,000 to 20,000 Daltons. In another embodiment, the molecular weight of the polymer as referred to herein is from 10,000 to 50,000 Daltons. In another embodiment, the molecular weight of the polymer as referred to herein is from 20,000 to 70,000 Daltons. In another embodiment, the molecular weight of the polymer as referred to herein is from 50,000 to 100,000 Daltons. In another embodiment, the molecular weight of the polymer as referred to herein is from 100,000 to 200,000 Daltons. In another embodiment, the molecular weight of the polymer as referred to herein is from 200,000 to 500,000 Daltons. In another embodiment, the molecular weight of the polymer as referred to herein is from 200,000 to 1,000,000 Daltons. In another embodiment, the molecular weight of the polymer as referred to herein is from 500,000 to 1,000,000 Daltons. In another embodiment, the molecular weight of the polymer as referred to herein is from 1,000,000 to 2,000,000 Daltons. Various molecular weight species have been shown to have the desired biological efficacy.

In one embodiment of this invention, low molecular weight Lipid-conjugates are defined hereinabove as the compounds of formula (I)-(XXI) wherein X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisacharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, hyaluronic acid, glycosaminoglycan, or polypyranose.

Examples of suitable divalent groups forming the optional bridging group Y are straight- or branched-chain alkylene, e.g., of 2 or more, preferably 4 to 18 carbon atoms, —CO-alkylene-CO, —NH-alkylene-NH—, —CO-alkylene-NH—, cycloalkylene, wherein alkylene in each instance, is straight or branched chain and contains 2 or more, preferably 2 to 18 carbon atoms in the chain, —(—O—CH(CH₃)CH₂—)ₓ— wherein x is an integer of 1 or more.

In another embodiment, in addition to the traditional phospholipid structure, related derivatives for use in this invention are phospholipids modified at the C1 or C2 position to contain an ether or alkyl bond instead of an ester bond. In one embodiment of the invention, the alkyl phospholipid derivatives and ether phospholipid derivatives are exemplified herein. In one embodiment, these derivatives are exemplified hereinabove by the general formulae (VIII) and (IX).

In one embodiment of the invention, X is covalently conjugated to a lipid. In another embodiment, X is covalently conjugated to a lipid via an amide bond. In another embodiment, X is covalently conjugated to a lipid via an esteric bond. In another embodiment, the lipid is phosphatidylethanolamine.

In one embodiment, cell surface GAGs play a key role in protecting cells from diverse damaging agents and processes, such as reactive oxygen species and free radicals, endotoxins, cytokines, invasion promoting enzymes, and agents that induce and/or facilitate degradation of extracellular matrix and basal membrane, cell invasiveness, white cell extravasation and infiltration, chemotaxis, and others. In addition, cell surface GAGs protect cells from bacterial, viral and parasitic infection, and their stripping exposes the cell to interaction and subsequent internalization of the microorganism. Enrichment of cell surface GAGs would thus assist in protection of the cell from injurious processes. Thus, in one embodiment of the invention, PLA2 inhibitors are conjugated to GAGs or GAG-mimicking molecules. In another embodiment, these Lipid-conjugates provide wide-range protection from diverse injurious processes, and ameliorate diseases that requires cell protection from injurious biochemical mediators.

In another embodiment, a GAG-mimicking molecule may be, inter alia, a negatively charged molecule. In another embodiment, a GAG-mimicking molecule may be, inter alia, a salicylate derivative. In another embodiment, a GAG-mimicking molecule may be, inter alia, a dicarboxylic acid.

In another embodiment, the invention provides a pharmaceutical composition for treating a subject suffering from cystic fibrosis, including a lipid or phospholipid moiety bonded to a physiologically acceptable monomer, dimer, oligomer, or polymer; and a pharmaceutically acceptable carrier or excipient.

In another embodiment, the invention provides a pharmaceutical composition for treating a subject suffering from cystic fibrosis, including any one of the compounds for use in the present invention or any combination thereof; and a pharmaceutically acceptable carrier or excipient. In another embodiment, the compounds for use in the present invention include, inter alia, the compounds represented by the structures of the general formulae as described hereinbelow: (A), (I), (II), (III), (IV), (V), (VI), (VII), (VIII), (IX), (IXa), (IXb), (X), (XI), (XII), (XIII), (XIV), (XV), (XVI), (XVII), (XVIII), (XIX), (XX), (XXI), (XXII), or any combination thereof.

### Preparation of Compounds for Use in the Present Invention

In one embodiment, the preparation of high molecular weight Lipid-conjugates for use in the methods of the present invention is as described in U.S. Pat. No. 5,064,817, which is incorporated fully herein by reference. In one embodiment, these synthetic methods are applicable to the preparation of low molecular weight Lipid-conjugates as well, i.e. Lipid-conjugates comprising monomers and dimers as the conjugated moiety, with appropriate modifications in the procedure as would be readily evident to one skilled in the art. The preparation of some low molecular weight Lipid-conjugates may be conducted using methods well known in the art or as described in U.S. Provisional Patent Application 60/704,874, which is incorporated herein by reference in its entirety.

### Dosages and Routes of Administration

The methods of this invention can be adapted to the use of the therapeutic compositions comprising Lipid-conjugates in admixture with conventional excipients, i.e. pharmaceutically acceptable organic or inorganic carrier substances suitable for parenteral, enteral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins, bronchodilators, steroids, anti-inflammatory compounds, gene therapy, i.e. sequences which code for the wild-type cystic fibrosis transmembrane conductance regulator (CFTR) receptor, surfactant proteins, etc., as will be understood by one skilled in the art.

In one embodiment, the invention provides for the administration of a salt of a compound as described herein as well. In one embodiment, the salt is a pharmaceutically acceptable salt, which, in turn may refer to non-toxic salts of compounds (which are generally prepared by reacting the free acid with a suitable organic or inorganic base) and include, but are not limited to, the acetate, benzenesulfonate, benzoate, bicarbonate, bisulfate, bitartrate, borate, bromide, calcium, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandlate, mesylate, methylbromide, methylnitrate, methylsulfate, mucate, napsylate, nitrate, oleate, oxalate, pamaote, palmitate, panthothenate, phosphate, diphospate, polygalacturonate, salicylate, stearate, subacetate, succinate, tannate, tartrate, teoclate, tosylate, triethiodide, and valerate salts, as well as mixtures of these salts.

In one embodiment, the route of administration may be parenteral, enteral, or a combination thereof. In another embodiment, the route may be intra-ocular, conjunctival, topical, transdermal, intradermal, subcutaneous, intraperitoneal, intravenous, intra-arterial, vaginal, rectal, intratumoral, parcanceral, transmucosal, intramuscular, intravascular, intraventricular, intracranial, inhalation, nasal aspiration (spray), sublingual, oral, aerosol or suppository or a combination thereof. In one embodiment, the dosage regimen will be determined by skilled clinicians, based on factors such as exact nature of the condition being treated, the severity of the condition, the age and general physical condition of the patient, etc.

In general, the doses utilized for the above described purposes will vary, but will be in an effective amount to exert the desired anti-disease effect. As used herein, the term “pharmaceutically effective amount” refers to an amount of a compound of formulae I-XXI which will produce the desired alleviation in symptoms or signs of disease in a patient. The doses utilized for any of the above-described purposes will generally be from 1 to about 1000 milligrams per kilogram of body weight (mg/kg), administered one to four times per day, or by continuous IV infusion. When the compositions are dosed topically, they will generally be in a concentration range of from 0.1 to about 10% w/v, administered 1-4 times per day.

In one embodiment, the use of a single chemical entity with potent anti-oxidant, membrane-stabilizing, anti-proliferative, anti-chemokine, anti-migratory, and anti-inflammatory activity provides the desired protection for a subject with CF, or in another embodiment, the methods of this invention provide for use of a combination of the compounds described. In another embodiment, the compounds for use in the present invention may be provided in a single formulation/composition, or in another embodiment, multiple formulations may be used. In one embodiment, the formulations for use in the present invention may be administered simultaneously, or in another embodiment, at different time intervals, which may vary between minutes, hours, days, weeks or months.

In one embodiment the compositions comprising the compounds for use in the present invention may be administered via different routes, which in one embodiment, may be tailored to provide different compounds at different sites, for example some compounds may be given parenterally to provide for superior perfusion throughout the lung and lymphatic system, and in another embodiment, some formulations/compounds/compositions may be provided via aerosol, or in another embodiment, intranasally, to provide for higher lung mucosal concentration.

In one embodiment, the compounds for use in the invention may be used for acute treatment of temporary conditions, or may be administered chronically, as needed. In one embodiment of the invention, the concentrations of the compounds will depend on various factors, including the nature of the condition to be treated, the condition of the patient, the route of administration and the individual tolerability of the compositions.

In one embodiment, the methods of this invention provide for the administration of the compounds in early life of the CF subject, or in another embodiment, throughout the life of the subject, or in another embodiment, episodically, in response to severity or constancy of symptomatic stages, or in another embodiment, at the onset of infection associated with CF, or in another embodiment, throughout infection in a subject with CF. In another embodiment, the patients to whom the lipid or PL conjugates should be administered are those that are experiencing symptoms of disease or who are at risk of contracting the disease or experiencing a recurrent episode or exacerbation of the disease, or pathological conditions associated with the same.

As used herein, the term “pharmaceutically acceptable carrier” refers to any formulation which is safe, and provides the appropriate delivery for the desired route of administration of an effective amount of at least one compound of the present invention. As such, all of the above-described formulations of the present invention are hereby referred to as “pharmaceutically acceptable carriers.” This term refers to as well the use of buffered formulations wherein the pH is maintained at a particular desired value, ranging from pH 4.0 to pH 9.0, in accordance with the stability of the compounds and route of administration.

For parenteral application, particularly suitable are injectable, sterile solutions, preferably oily or aqueous solutions, as well as suspensions, emulsions, or implants, including suppositories. Ampoules are convenient unit dosages.

For application by inhalation, particularly for treatment of airway obstruction or congestion, solutions or suspensions of the compounds mixed and aerosolized or nebulized in the presence of the appropriate carrier suitable.

For topical application, particularly for the treatment of skin diseases such as contact dermatitis or psoriasis, admixture of the compounds with conventional creams or delayed release patches is acceptable.

For enteral application, particularly suitable are tablets, dragees, liquids, drops, suppositories, or capsules. A syrup, elixir, or the like can be used when a sweetened vehicle is employed. When indicated, suppositories or enema formulations may be the recommended route of administration.

Sustained or directed release compositions can be formulated, e.g., liposomes or those wherein the active compound is protected with differentially degradable coatings, e.g., by microencapsulation, multiple coatings, etc. It is also possible to freeze-dry the new compounds and use the lyophilisates obtained, for example, for the preparation of products for injection.

It will be appreciated that the actual preferred amounts of active compound in a specific case will vary according to the specific compound being utilized, the particular compositions formulated, the mode of application, and the particular situs and organism being treated. Dosages for a given host can be determined using conventional considerations, e.g., by customary comparison of the differential activities of the subject compounds and of a known agent, e.g., by means of an appropriate, conventional pharmacological protocol.

### Methods of Preventing or Treating CF Using PL Conjugates

In one embodiment of the invention, the methods of the present invention make use of a compound as described herein to treat a subject suffering from cystic fibrosis, reduce or delay the mortality of a subject suffering from cystic fibrosis or ameliorate symptoms associated with cystic fibrosis.

In one embodiment, the compound for use in the present invention comprises dipalmitoyl phosphatidylethanolamine and heparin. In one embodiment, the compound for use in the present invention comprises dipalmitoyl phosphatidylethanolamine and chondroitin sulfate. In one embodiment, the compound for use in the present invention comprises dipalmitoyl phosphatidylethanolamine and hyaluronic acid. In one embodiment, the compound for use in the present invention comprises dipalmitoyl phosphatidylethanolamine and carboxymethylcellulose. In one embodiment, the compound for use in the present invention comprises dimyristoyl phosphatidylethanolamine and hyaluronic acid.

In one embodiment, the compound for use in the present invention is a dipalmitoyl phosphatidylethanolamine conjugated via an amide or ester bond to a glycosaminoglycan. In one embodiment, the compound for use in the present invention is a dipalmitoyl phosphatidylethanolamine conjugated via an amide or ester bond to a chondroitin sulfate, which is chondroitin-6-sulfate, chondroitin-4-sulfate or a derivative thereof. In another embodiment, the compound for use in the present invention is a dipalmitoyl phosphatidylethanolamine conjugated via an amide or ester bond to a heparin. In another embodiment, the compound for use in the present invention is a dipalmitoyl phosphatidylethanolamine conjugated via an amide or ester bond to a hyaluronic acid. In another embodiment, the compound for use in the present invention is a dimyristoyl phosphatidylethanolamine conjugated via an amide or ester bond to a hyaluronic acid.

In one embodiment, the lipid-conjugates display a wide-range combination of cytoprotective pharmacological activities, which are useful in the present invention. In one embodiment, the compounds may be useful for their anti-inflammatory effects, as the inflammatory process itself may be partially or mostly responsible for lung damage in cystic fibrosis. Cellular elaboration of cytokines and chemokines serve an important regulatory function in health; however, when a hyperactive response to stress or disease is triggered, these compounds may present in excess and damage tissue, thereby pushing the disease state toward further deterioration. In one embodiment, the lipid compounds for use in the methods of this invention, possess a combination of multiple and potent pharmacological effects, including inter-alia the ability to inhibit the extracellular form of the enzyme phospholipase A2.

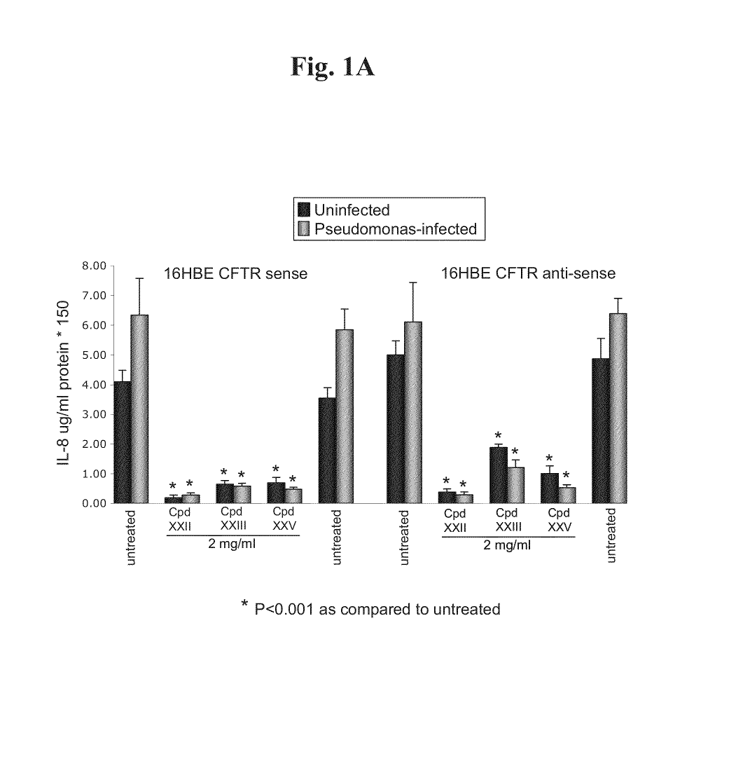

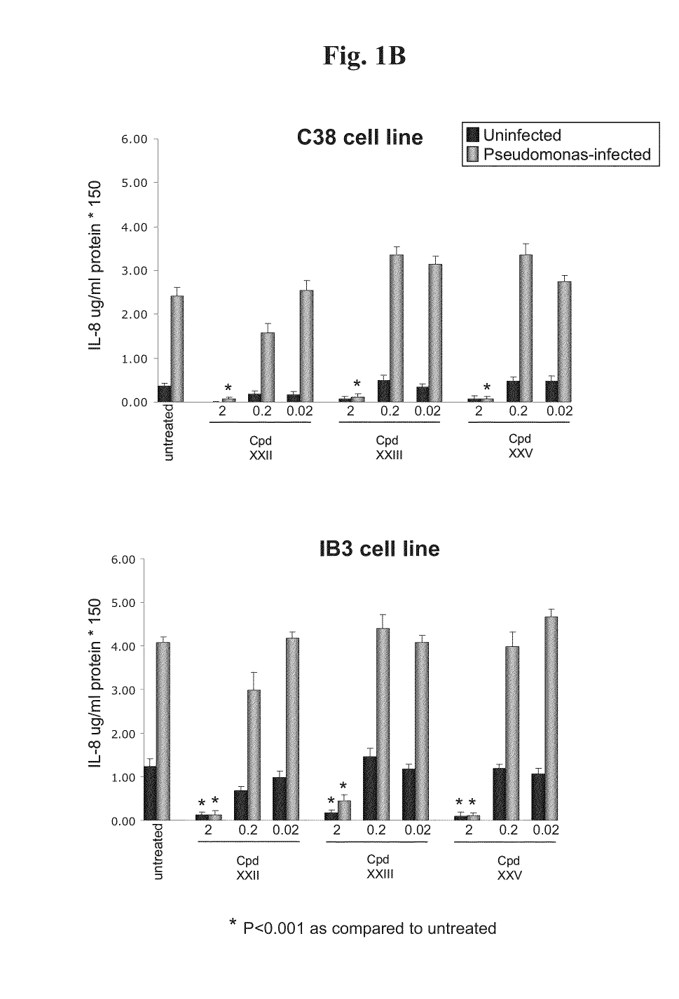

In one embodiment, inflammation is a primary effect of CF, while in another embodiment, inflammation is due to a secondary effect, which in one embodiment is infection, to which subjects with cystic fibrosis are more susceptible. In one embodiment, the infection is Pseudomonas infection. In another embodiment, the compounds for use in the present invention may be useful for their anti-inflammatory effects in bronchial epithelial cells, as well as in Pseudomonas-infected bronchial cells, which is exemplified, in one embodiment, in FIG. 1.

In one embodiment, lipid-conjugates are useful in affecting inflammation in a subject with cystic fibrosis, where the subject is administered lipid-conjugates at presymptomatic stages of the disease. A characteristic feature of inflammation in the CF lung is the persistent infiltration of massive numbers of neutrophils into the airways. Although neutrophils help to control infection, when present in great excess, they can be harmful. Major advances in the understanding of the inflammatory process in the CF lung have come from the use of bronchoscopy and bronchoalveolar lavage (BAL) to analyze the inflammatory process in patients who are relatively symptom free and/or do not regularly produce sputum. Recent BAL studies suggest that neutrophil-rich inflammation begins very early, even in infants without clinically apparent lung disease. Thus, in one embodiment, the compounds of the present invention may be useful in treating CF, even in presymptomatic stages of disease.

In one embodiment, the lipid-conjugates affect an underlying bias toward inflammation in a subject with CF, irrespective of exposure to traditional inflammatory stimuli. This is exemplified in one embodiment in FIG. 1 by a reduction of increased baseline IL-8 levels in non-Pseudomonas-infected cells treated with Lipid-conjugates.

A number of chemoattractants from epithelial cells, macrophages, neutrophils themselves, and bacterial products contribute to the neutrophil influx in CF subjects. Some infants have inflammation even in the apparent absence of infection, leading to the speculation that inflammation may precede infection in CF. According to this aspect of the invention, and in one embodiment, the methods of the invention may be useful, in particular, in suppressing inflammatory responses in a subject with CF, either prior to or following infection, which may, in another embodiment, be accompanied by inflammatory responses.

Links between the basic defect in CF and inflammation may exist, in other embodiments, with dysregulation of cytokine production and abnormal epithelial host defenses being causal factors of sustained inflammation. Regardless of the details of how this process is initiated and/or perpetuated, in other embodiments, inflammation beginning at a very early stage and/or progressing throughout the life of the CF subject may be alleviated, treated, prevented, inhibited, mitigated or otherwise positively affected via the methods and uses of the compounds described in the present invention.

Subjects with CF may be those with faulty or absent “cystic fibrosis transmembrane conductance regulator (CFTR) function or activity”, which in turn, is marked by aberrant function, in comparison to the function or activity of that normally performed by wild-type CFTR. Such functions can include mediation, regulation or control of ion, (e.g. chloride (Cl—) ion) transport across cellular membranes.

A subject with CF, in turn, may have CF-defective or affected cells, which lack cystic fibrosis transmembrane conductance regulator function, either due to the absence of CFTR, or due to a CFTR mutant polypeptide that is unable to provide CFTR function and/or activity, or is less effective in providing CFTR function and/or activity. Examples of such cells include CFTR mutants (e.g., CFTR ΔF508) of which at least 1300 different varieties have been identified. See, for example, Kunzelmann et al, “Pharmacotherapy of the Ion Transport Defect in Cystic Fibrosis,” Clin. Exper. Pharm. Phys. (2001) 28:857-67; Welsh et al, “Molecular Mechanisms of CFTR Chloride Channel Dysfunction in Cystic Fibrosis,” Cell (1993) 73:1251-54. In one embodiment, CFTR mutations result in improper trafficking of the receptor to the cell membrane. Such a subject may benefit from the methods of this invention. In one embodiment, a defective CFTR leads to defects in ion transport across a cell membrane, which in one embodiment leads to increased levels of mucin, which in one embodiment triggers an anti-inflammatory response. In another embodiment, a defective CFTR leads to dysregulated cytokine production by neutrophils.

Administration of the Lipid-conjugates provide, in another embodiment, cytoprotective effects, which are useful in the treatment of CF, or infection/inflammation associated with CF. The compounds, in some embodiments, are able to stabilize biological membranes; inhibit cell proliferation; suppress free radical production; suppress nitric oxide production; reduce cell migration across biological bathers; influence chemokine levels, including MCP-1, ENA-78, Gro α, and CX3C; influence cytokine levels, including IL-6 and IL-8; affect gene transcription and modify the expression of MHC antigens; bind directly to cell membranes and change the water structure at the cell surface; prevent airway smooth muscle constriction; reduce expression of tumor necrosis factor-α (TNF-α); modify expression of transcription factors such as NFκB; and inhibit extracellular degradative enzymes, including collagenase, heparinase, hyaluronidase, in addition to that of PLA2.

In one embodiment, the compounds for use in the methods of the present invention treat CF through exerting at least one of their many pharmacological activities, among which are amelioration, or prevention, of tissue injury arising in the course of pathological disease states by stabilizing cell membranes; limiting oxidative damage; limiting cell proliferation, cell extravasation; suppressing immune responses; or attenuating physiological reactions to stress, as expressed in elevated chemokine levels. In one embodiment of the present invention, the useful pharmacological properties of the lipid or Lipid-conjugates may be applied for clinical use, and disclosed herein as methods for treatment of a disease. The biological basis of these methods may be readily demonstrated by standard cellular and animal models of disease as known in the art, and as described below.

In one embodiment, the Lipid-conjugates provide far-reaching cytoprotective effects to an individual suffering from CF wherein one or more of the presiding pathophysiological mechanisms of tissue damage entail either oxidation insult giving rise to membrane fragility; excessive expression of chemokines and cytokines associated with tissue damage; cell membrane damage; excessive nitric oxide production giving rise to lung tissue insult, etc.

In one embodiment, the administration of Lipid-conjugates provides a method for decreasing the expression of proinflammatory chemokines, cytokines, or a combination thereof. In another embodiment, the administration of Lipid-conjugates provides a method of affecting endogenous activation of NF-κB, IL-6 and IL-8 in human airway epithelial cell lines.

While pharmacological activity of the Lipid-conjugates described herein may be due in part to the nature of the lipid moiety, the multiple and diverse combination of pharmacological properties observed for the Lipid-conjugates may represent, in other embodiments, the ability of the compound to act essentially as several different drugs in one chemical entity. Thus, for example, lung mucosal or lung parenchymal injury, as may occur in CF, may be attenuated by any one or all of the pharmaceutical activities of immune suppression, anti-inflammation, anti-oxidation, suppression of nitric oxide production, or membrane stabilization.

In one embodiment, the invention provides a method of “treating” CF or related diseases or disorders, which in one embodiment, refers to both therapeutic treatment and prophylactic or preventative measures, wherein the object is to prevent or lessen the targeted pathologic condition or disorder as described hereinabove. In one embodiment, treating refers to delaying the onset of symptoms, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, preventing relapse to a disease, decrease the number or frequency of relapse episodes, increasing latency between symptomatic episodes, increasing time to sustained progression, expediting remission, inducing remission, augmenting remission, speeding recovery, or increasing efficacy of or decreasing resistance to alternate therapeutics.

Thus, in one embodiment, the invention provides methods for treating a subject suffering from cystic fibrosis, reducing or delaying the mortality of a subject suffering from cystic fibrosis or ameliorating symptoms associated with cystic fibrosis, and the compounds/compositions/formulations, in one embodiment, diminish or abrogate a deleterious inflammatory response in said subject, or in another embodiment, prevent, treat, reduce the incidence of, reduce the severity of, delay the onset of, or diminish the pathogenesis of an infection is the CF subject. In another embodiment, the invention provides methods for decreasing expression of proinflammatory chemokines, cytokines, or a combination thereof, while in another embodiment, the invention provides methods of activating NF-κB, IL-6, IL-8, or a combination thereof in human airway epithelial cell lines.

In one embodiment, symptoms are primary, while in another embodiment, symptoms are secondary. In one embodiment, “primary” refers to a symptom that is a direct result of faulty or absent CFTR expression, or in another embodiment, ‘secondary” refers to a symptom that is derived from or consequent to a primary cause, such as, for example, infection with a pathogen. In another embodiment, symptoms may be any manifestation of a disease or pathological condition, comprising inflammation, swelling, fever, pain, bleeding, itching, runny nose, coughing, headache, migraine, difficulty breathing, weakness, fatigue, drowsiness, weight loss, nausea, vomiting, constipation, diarrhea, numbness, dizziness, blurry vision, muscle twitches, convulsions, etc., or a combination thereof.

In one embodiment, the methods are useful in treating an infection in a subject, wherein the pathogen is a virus or in another embodiment, the pathogen is a bacterium. In one embodiment, the infection is with a pathogen which infects the respiratory system, such as mycobacteria, pseudomonas, cryptococcus, streptococcus, reovirus, influenza, or other infections known to those of skill in the art.

Typically, subjects with CF are afflicted with Staphylococcus aureus which early in life is the pathogen most often isolated from the respiratory tract, but as the disease progresses, Pseudomonas aeruginosa is most frequently isolated. A mucoid variant of Pseudomonas is uniquely associated with CF. Colonization with Burkholderia cepacia occurs in up to 7% of adult patients and may be associated with rapid pulmonary deterioration. Treatment of a subject with infection with any of these agents is to be considered as part of this invention.

Treatment includes prevention of airway obstruction and prophylaxis against and control of pulmonary infection, which may be effected via the methods and using the compounds/compositions of this invention. Prophylaxis against pulmonary infections may be accomplished via the compounds/compositions of this invention, and may include maintenance of pertussis, Haemophilus influenzae, varicella, and measles immunity and may be combined with immunization against the same and other respiratory infections in particular, in combination with annual influenza vaccination, or in another embodiment, in conjunction with amantadine prophylaxis against influenza A.

The methods of this invention may also be in combination with chest physical therapy consisting of postural drainage, percussion, vibration, and assisted coughing, as known in the art. In older patients, alternative airway clearance techniques such as active cycle of breathing, autogenic drainage, flutter valve device, positive expiratory pressure mask, and mechanical vest therapy may be effective. For reversible airway obstruction, bronchodilators may be given orally and/or by aerosol and corticosteroids by aerosol. O₂ therapy is indicated for patients with severe pulmonary insufficiency and hypoxemia, and may accompany administration of the compounds/compositions of this invention.

Mechanical ventilation may be used in combination therapy for the methods of this invention, in another embodiment, and in one embodiment, it should be restricted to patients with good baseline status in whom acute respiratory failure develops, in association with pulmonary surgery, or in patients awaiting lung transplantation who develop hypercapnic respiratory failure. Noninvasive positive pressure ventilation by nasal or face mask also can be beneficial and can be accomplished in conjunction with therapy with the compounds/compositions of this invention.

Oral expectorants may also be administered in conjunction with the compounds/compositions of this invention. Long-term daily aerosol administration of dornase alfa (recombinant human deoxyribonuclease) has been shown to slow the rate of decline in pulmonary function and to decrease the frequency of severe respiratory tract exacerbations, and may be used accordingly.

Oral corticosteroids are indicated in infants with prolonged bronchiolitis and in those patients with refractory bronchospasm, allergic bronchopulmonary aspergillosis, and inflammatory complications (eg, arthritis and vasculitis), and may be used in combination with the compounds/compositions of this invention.

CTLA4-Ig fusion protein, which in one embodiment is Abatacept, and in one embodiment modulates the T cell co-stimulatory signal mediated through the CD28-CD80/86 pathway, may also be used in combination with the compounds/compositions of this invention.

Ibuprofen, when given at a dose sufficient to achieve a peak plasma concentration between 50 and 100 μg/mL over several years, has been shown to slow the rate of decline in pulmonary function, especially in children 5 to 13 yr, and may accompany the administration of the compounds/compositions of this invention.

Antibiotics should be used in symptomatic patients to treat bacterial pathogens in the respiratory tract, according to culture and sensitivity testing. A penicillinase-resistant penicillin (eg, cloxacillin or dicloxacillin) or a cephalosporin (eg, cephalexin) is the drug of choice for staphylococci. Erythromycin, amoxicillin-clavulanate, ampicillin, tetracycline, trimethoprim-sulfamethoxazole, or occasionally chloramphenicol may be used individually or in combination for protracted ambulatory therapy of pulmonary infection due to a variety of organisms. Ciprofloxacin is effective against sensitive strains of Pseudomonas. For severe pulmonary exacerbations, especially in patients colonized with Pseudomonas, parenteral antibiotic therapy is advised, often requiring hospital admission but safely conducted at home in carefully selected patients. Combinations of an aminoglycoside (tobramycin, gentamicin) with an anti-Pseudomonas penicillin are given IV. Intravenous administration of cephalosporins and monobactams with anti-Pseudomonas activity also may be useful. Serum aminoglycoside concentrations should be monitored and dosage adjusted to achieve a peak level of 8 to 10 μg/mL (11 to 17 μmol/L) and a trough value of <2 μg/mL (<4 μmol/L). The usual starting dose of tobramycin or gentamicin is 7.5 to 10 mg/kg/day in 3 divided doses, but high doses (10 to 12 mg/kg/day) may be required to achieve acceptable serum concentrations. Because of enhanced renal clearance, large doses of some penicillins may be required to achieve adequate serum levels. It is to be understood that administration of the compounds/compositions of this invention may be in conjunction with any antibiotic, and the invention is exemplified with the guidelines presented herein, but is by no means restricted to these examples.

In another embodiment, aerosol therapy with ribavirin may be used in combination with the compounds/compositions of this invention for combatting viral infection, in particular, in one embodiment, in infants with CF and presenting with RSV infection.

Surgery may be indicated for localized bronchiectasis or atelectasis that cannot be effectively treated medically; nasal polyps; chronic sinusitis; bleeding from esophageal varices secondary to portal hypertension; gallbladder disease; and intestinal obstruction due to a volvulus or an intussusception that cannot be medically reduced. Any of these procedures may be accompanied by the administration of the compounds/compositions of this invention, at any point, prior to, during or following the procedure, or with any combination thereof, and is to be considered as part of this invention.

Thus, in one embodiment of the present invention, the compounds of the present invention are directed towards resolution of symptoms of the disease or disorder that result from a pathogenic infection as described hereinabove. In another embodiment, the compounds affect the pathogenesis underlying the pathogenic effect described hereinabove.

In one embodiment of the invention, the treatment requires controlling the expression production and activity of phospholipase enzymes. In another embodiment, the treatment requires controlling the production and/or action of lipid mediators. In another embodiment, the treatment requires amelioration of damage to glycosaminoglycans (GAG) and proteoglycans. In another embodiment, the treatment requires controlling the production and action of oxidants, oxygen radicals and nitric oxide. In another embodiment, the treatment requires anti-oxidant therapy. In another embodiment, the treatment requires anti-endotoxin therapy. In another embodiment, the treatment requires controlling the expression, production or action of cytokines, chemokines, adhesion molecules or interleukins. In another embodiment, the treatment requires protection of lipoproteins from damaging agents. In another embodiment, the treatment requires controlling the proliferation of cells. In another embodiment, the treatment requires inhibition of invasion-promoting enzymes. In another embodiment, the treatment requires controlling cell invasion. In another embodiment, the invading cells are white blood cells. In another embodiment, the treatment requires controlling white cell activation, adhesion or extravasation. In another embodiment, the treatment requires inhibition of lymphocyte activation. In another embodiment, the treatment requires controlling of blood vessel and airway contraction. In another embodiment, the treatment requires tissue preservation.

In one embodiment of the invention, the lipid mediator is a glycerolipid. In another embodiment, the lipid mediator is a phospholipid. In another embodiment, the lipid mediator is sphingolipid. In another embodiment, the lipid mediator is a sphingosine. In another embodiment, the lipid mediator is ceramide. In another embodiment, the lipid mediator is a fatty acid. In another embodiment, the fatty acid is arachidonic acid. In another embodiment, the lipid mediator is an arachidonic acid-derived eicosanoid. In another embodiment, the lipid mediator is a platelet activating factor (PAF). In another embodiment, the lipid mediator is a lysophospholipid.

In one embodiment of the invention, the damaging agent is a phospholipase. In another embodiment, the damaging agent is a reactive oxygen species (ROS). In another embodiment, the damaging agent is a free radical. In another embodiment, the damaging agent is a lysophospholipid. In another embodiment, the damaging agent is a fatty acid or a derivative thereof. In another embodiment, the damaging agent is hydrogen peroxide. In another embodiment, the damaging agent is a phospholipid. In another embodiment, the damaging agent is an oxidant. In another embodiment, the damaging agent is a cationic protein. In another embodiment, the damaging agent is a streptolysin. In another embodiment, the damaging agent is a protease. In another embodiment, the damaging agent is a hemolysin. In another embodiment, the damaging agent is a sialidase.

In one embodiment of the invention, the invasion-promoting enzyme is collagenase. In another embodiment, the invasion-promoting enzyme is matrix-metaloproteinase (MMP). In another embodiment, the invasion-promoting enzyme is heparinase. In another embodiment, the invasion-promoting enzyme is heparanase. In another embodiment, the invasion-promoting enzyme is hyaluronidase. In another embodiment, the invasion-promoting enzyme is gelatinase. In another embodiment, the invasion-promoting enzyme is chondroitinase. In another embodiment, the invasion-promoting enzyme is dermatanase. In another embodiment, the invasion-promoting enzyme is keratanase. In another embodiment, the invasion-promoting enzyme is protease. In another embodiment, the invasion-promoting enzyme is lyase. In another embodiment, the invasion-promoting enzyme is hydrolase. In another embodiment, the invasion-promoting enzyme is a glycosaminoglycan degrading enzyme. In another embodiment, the invasion-promoting enzyme is a proteoglycan degrading enzyme.

In one embodiment of the invention, the term “controlling” refers to inhibiting the production and action of any of the factors ad herein described in order to maintain their activity at the normal basal level and suppress their activation in pathological conditions.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

The compounds for use in the instant invention are collectively referred to as Lipid-conjugates.

### Example 1

### Glycolipid Conjugates Modulate Chemokine and/or Cytokine Expression in CF Airway Epithelial Cells In Vitro

The effects of the Lipid-conjugates were tested in the following cell lines: 16HBE, IB-3 and C-38 cells.

The 16HBE cells are a well-characterized human bronchial epithelial cell line which form tight junctions and have been extensively used in the analysis of CF airway inflammation. When transfected with a vector encoding CFTR in the antisense orientation they provide a well characterized model for CF as compared with the same cells expressing CFTR in the sense orientation. As transfection itself activates NF-kB, it is important to use equivalent controls to test effects of a drug on proinflammatory signaling.

IB-3 and C-38 are a CF (which may include a vector control) and “corrected” cell line. IB-3 cells were created in 1992 from primary culture of bronchial epithelia cells isolated from a CF patient. The CF phenoptype was corrected in C-38 cell line by transfection with wild-type adeno-associated viral CFTR, allowing the cells to stably express wild-type CFTR. These lines have been used extensively in comparisons of CF and control cells.

The cells were grown to confluency in 96 well plates, washed, and Lipid-conjugates (Compounds XXII, XXIII, and XXV) or sham were added to the cells, which were incubated at 37° C. for 30 minutes. Cells were washed, and in some groups, incubated with heat-killed P. aeruginosa PAO1 (5×10⁷ cfu/ml) for 24 hours. Cells were then washed extensively and incubated in fresh media containing gentamicin (100 μg/ml). Supernatants were then harvested, and IL-8 levels were assayed by ELISA. The data was analyzed for statistical significance using an ANOVA.

Data presented in FIG. 1 demonstrate that Lipid-conjugates significantly and dose-dependently suppress IL-8 expression in both mutant CFTR and control cell lines (FIGS. 1A and 1B). Further, IL-8 suppression by Lipid-conjugates is present both in cells exposed to PAO1 and in uninfected cells (FIGS. 1A and 1B). Additionally, Lipid-conjugates inhibit endogenous IL-8 production associated with mutant CFTR. Thus, Lipid-conjugates may be useful in decreasing inflammatory symptoms in CF patients, both those that are suffering from an infection and those that are not.

The levels of other chemokines and cytokines in the cell supernatants are determined by ELISA as described hereinabove.

In order to determine whether NF-kB activation occurs in the sham versus treated cells, cells are transfected with a NF-kB luciferase construct using Fugene. 24 hours following transfection, cells are weaned from serum, incubated for 18 hours, then treated with the compounds, or sham, respectively. Additional groups include cells infected with PAO1 for 60 minutes, then processed as described. Cell lysates are screened for luciferase activity.

Effects of Lipid-conjugates on the activation of other transcription factors that may be relevant to airway disease in CF may be similarly evaluated, via construction of luciferase constructs, via methods known in the art. Microarrays for screening for effects of the compounds on multiple proinflammatory genes, versus sham treated cells, may also be evaluated.

The effect of Lipid-conjugates on human airway epithelial cells in primary culture is evaluated as well, for example, probing isolated nasal polyp tissue.

### Example 2

### Immobilized Phosphatidylethanolamine (PE) Inhibitors of Extracellular PLA2

Polysaccharide-immobilized phosphatidylethanolamine (PE) provided the following results:

| | | | |
|---|---|---|---|
| MK645, | Hyaluronic acid/ | av MW = 50-200 | K_{1/2} = kill |
| | PE; | kDa. | |
| MK 723/4, | Hemacell/PE, | av. MW = 30 kDa. | K_{1/2} = 5 μM |
| MK691, | Chondroitin SO₄/ | av. MW ~50 kDa. | K_{1/2} => 1 μM, kill |
| | PE, | | |
| MK713/4 | Dextran/PE | av. MW = 40 kDa. | K_{1/2} => 30 μM |
| MK714/1 | Dextran/PE | av. MW = 40 kDa. | K_{1/2} = 4 μM |

Samples were prepared at 20 mg/ml in PBS buffer, and were suspended by vigorous vortexing, shaking at 37° C., and “tip” or bath sonicated for 20 seconds. MK723/4 dissolved easily. The others compounds proved more difficult to dissolve, but ultimately did using these conditions.

The compounds were assessed for their ability to inhibit IL-8 secretion from IB3-1 cells, with the most potent compound being MK714/1. Based on the calculated PE content, the K_{1/2} was estimated to be roughly 4 μM. The order of activity was:

### MK714/1>MK723/4>MK713/4>>[MK645, MK691].

The values of K_{1/2} given in the table are calculated from the concentration of PE's on each molecule of carrier polysaccharide rather than on mg/ml of each complex adduct.

MK645 (at 1 mg/ml) and MK723/4 (at 0.2 mg/ml) were found to be toxic to IB3-1 cells when incubated for 24 hours, while the other compounds were not.

### Example 3

### Glycolipid Conjugates Modulate Modulate Chemokine and/or Cytokine Expression in CF Mouse Models In Vivo

The following mouse models of CF are known in the art, and may be used to evaluate positive effects of the compounds of this invention on CF pathogenesis.

Knockout mice genetically disrupted for the CF gene, as described by Snouwaert et al [Science 1992; 257:1083-1088], Ratcliff et al. [Genet 1993; 4:35-41], O'Neal et al. [Hum Mol Genet 1993; 2:1561-1569], Hasty et al. [Somat Cell Mol Genet 1995; 21:177-187], or mice with a AF508 mutation, such as described by Colledge et al. [Nat Genet 1995; 10:445-452], Zeiher et al. [J Clin Invest 1995; 96:2051-2064], van Doorninck et al [Embo J 1995; 14:4403-4411], and others may be used.

Compounds of the invention are administered to the animals, and effects on cytokine and chemokine production are measured as a function of time. Animal responses to challenge with infection with bacteria, such as Psuedomonas species are evaluated, as well.

Affymetrix mouse gene arrays may be used to detect differential expression (relative intensity plotted on y-axis v. pairs of mice of increasing age on x-axis) of lung mRNAs isolated from age-matched wild-type and CFTR-deficient mice, for example CFTR(+/+) versus FABP-hCFTR/mCFTR(−/−) or CFTR(−/−) mice. A CFTR-deficient mouse expressing mutated CFTR, SPC-hA508/FABP-hCFTR/mCFTR(−/−), may also analyzed in the same manner, as well as mice with other mutations to the CFTR gene, including doxycycline-induced mutations. Evaluation of genes, which can potentially modify CFTR-dependent pathways, and therefore, the CF disease process may be conducted prior to and over the course of treatment with a given compound, or combinations of compounds. Positive effects in terms of disease severity, in terms, inter-alia of susceptibility and response to infection may be evaluated. Mouse lung RNA may be harvested and assessed for changes in gene expression, using such arrays. CFTR-dependent defects in chloride (Cl⁻) transport and cell function may be assessed in this context, as well.

Human CFTR cDNA is expressed in the intestinal epithelium under control of the intestinal fatty acid binding protein gene promoter (iFABP), fully correcting small intestinal pathology and supporting normal postnatal survival of CFTR (−/−) transgenic mice. The iFABP-hCFTR, CFTR (−/−) mice can be maintained in a mixed FVB/N, C57BL/6 background without evidence of GI or pulmonary disease. Histological and biochemical studies identify no overt pathology in lung tissue from these mice compared to CFTR-expressing littermate controls. See Zhou et al, Science, (1994), 266:1705-8; Chroneos, J. Immunol., (2000) 165:3941-50. Mice are housed in microisolator cages. Lungs of adult iFABP-hCFTR, CFTR (−/−) and control mice are free of bacterial pathogens or colonization as assessed by quantitative culture of lung homogenates on blood agar plates.

Matings of FABP-hCFTR (+/+)/mCFTR (−/−) mice to wild type FVB/N-mCFTR (+1+) mice, are used to produce F1 FABP-hCFTR (±)/mCFTR (∓) mice. These mice are crossed to generate F2 offspring littermates which are then genotyped. Genotyping is performed using the following primers: primers for mCFTR PCR are forward primer (intron 9): 5′-AGG GGC TCG CTC TTC TTT GTG AAC, -3′ reverse primer (intron 10): 5′-TGG CTG TCT GCT TCC TGA CTA TGG, -3′ for neomycin resistance gene PCR are forward primer: 5′-CAC AAC AGA CAA TCG GCT GCT, -3′ and reverse primer: 5′-ACA GTT CGG CTG GCG CGA G, -3′ and for hCFTR PCR are forward primer (exon 9): 5′-AAA CTT CTA ATG GTG ATG ACA G-3′. Reverse primer (exon 11): 5′-AGA AAT TCT TGC TCG TTG AC-3′. FABP-hCFTR(+/+)/mCFTR (−/−) and hCFTR (+/+)/mCFTR (+/+) mice are identified. All CFTR (+/+) mice are heterozygous for the targeted mCFTR gene.

The effects of compound use in these mice in terms of their susceptibility to infection, mortality, etc., is assessed, further in response to administration of a compound or compounds of the invention.

### Example 4

### Glycolipid Conjugates Modulate Airway Inflammation During P. aeruginosa Infection In Vivo

I.P. Glycolipid Conjugate treatment:

Five day-old C57BL6 mice (average weigh 3.5 g, 6/group) receive one of three doses of glycolipid conjugates via i.p. injection at −18 h, −0.5 h and +4 h after P. aeruginosa or PBS (control) injection.

### Aerosolized Glycolipid Conjugate Treatment:

Five day-old C57BL6 mice receive 1 mg/kg aerosolized Compound XXII (treatment group) or an equivalent volume of aerosolized PBS (control) at −18 h and +0.5 h after P. aeruginosa or PBS (control) infection.

In a separate experiment, conjugate-treated and non-treated mice are intranasally inoculated with 1-5×10⁸ cfu of P. aeruginosa in 10 μl of PBS or PBS alone (control) on day 6.

On day seven, mice are sacrificed, and lungs homogenized using 40 μM cell strainers (BD Falcon) to obtain single-cell suspensions. Bacterial counts in lung and spleen are determined and the percentage of mice that develop pneumonia (defined as >1000 cfu/lung and histopathology compatible with lung inflammation) or bacteremia (>5 cfu/spleen) determined. The percentage of Polymorphonuclear Neutrophils (PMNs) among total leukocytes is determined by surface staining of Ly-6G (PMNs) and CD45 (leukocytes) and flow cytometry analysis.

### Example 5

### Glycolipid Conjugates Modulate Inflammatory Cytokine Expression in Humans In Vivo

Broncheoalveolar lavage (BAL) fluids are obtained from CF patients, and age and gender matched controls. Assays for cytokine expression are conducted as in Example 1, for example via ELISA assay. Baseline expression levels are compared to those obtained following administration of the compounds, in particular following treatment with Compound XXII, XXIII, XXIV or XXV.

CF patients frequently suffer from infection with Pseudomonas aeruginosa which are isolated from sputum samples, as well. Sputum is collected at baseline and following treatment as above, bacterial counts are assessed, as well as symptoms and other indicators of disease.

What we claim is:

1. A method for treating a subject suffering from cystic fibrosis, reducing or delaying the mortality of a subject suffering from cystic fibrosis, or ameliorating symptoms associated with cystic fibrosis, the method comprising the step of administering a therapeutically effective amount of a compound represented by the structure of the general formula (A): 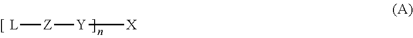
wherein
L is a phospholipid;
Z is ethanolamine;
Y is either nothing or a spacer group ranging in length from 2 to 30 atoms;
X is a glycosaminoglycan; and
n is a number from 1 to 1000;
wherein any bond between L, Z, Y and X is either an amide or an esteric bond to a subject suffering from symptoms of cystic fibrosis.

2. A method for treating a subject suffering from cystic fibrosis, reducing or delaying the mortality of a subject suffering from cystic fibrosis or ameliorating symptoms associated with cystic fibrosis, the method comprising the step of administering therapeutically effective amount of a compound represented by the structure of the general formula (I): 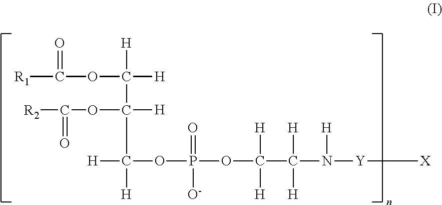
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a glycosaminoglycan; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond to a subject suffering from symptoms of cystic fibrosis.

3. The method of claim 1, wherein said compound is represented by the structure of the general formula (VIII): 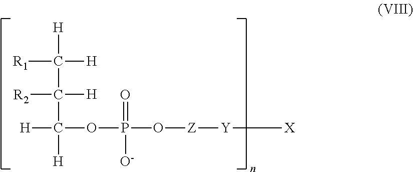
wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is ethanolamine;
Y is either nothing or a spacer group ranging in length from 2 to 30 atoms;
X is a glycosaminoglycan; and
n is a number from 1 to 1000;
wherein any bond between the phospholipid, Z, Y and X is either an amide or an esteric bond.

4. The method of claim 1, wherein said compound is represented by the structure of the general formula (IX): 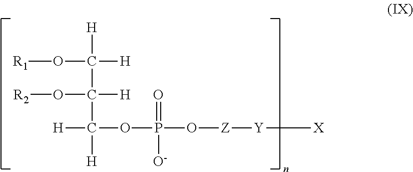
wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is ethanolamine;
Y is either nothing or a spacer group ranging in length from 2 to 30 atoms;
X is a glycosaminoglycan; and
n is a number from 1 to 1000;
wherein any bond between the phospholipid, Z, Y and X is either an amide or an esteric bond.

5. The method of claim 1, wherein said compound is represented by the structure of the general formula (X): 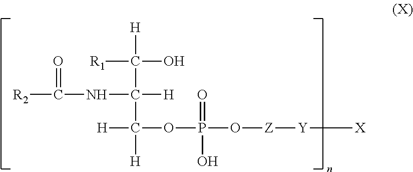
wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is ethanolamine;
Y is either nothing or a spacer group ranging in length from 2 to 30 atoms;
X is a glycosaminoglycan; and
n is a number from 1 to 1000;
wherein any bond between the ceramide phosphoryl, Z, Y and X is either an amide or an esteric bond.

6. The method according to anyone of claims 1-5, wherein said glycosaminoglycan is hyaluronic acid, heparin, heparin sulfate, chondroitin sulfate, keratin, keratin sulfate or dermatan sulfate.

7. The method according to anyone of claims 1-5, wherein L is palmitoyl phosphatidylethanolamine or myristoyl phosphatidylethanolamine.

8. The method according to anyone of claims 1-5, wherein said n is a number between 2 to 100.
